(19) United States
(12) Patent Application Publication
(10) Pub. No.: US 2009/0285770 A1
(43) Pub. Date: Nov. 19, 2009

(54) Topical Compositions for Promoting Homeostasis of the Skin

(75) Inventor: Julien Laboureau, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'Oreal, Paris (FR)

(21) Appl. No.: 12/085,228

(22) PCT Filed: Nov. 17, 2006
(86) PCT No.: PCT/EP2006/068629

§ 371 Date: Jul. 14, 2008

Related U.S. Application Data
(60) Provisional application No. 60/741,908, filed on Dec. 5, 2005.

(30) Foreign Application Priority Data
Nov. 21, 2005 (FR) 0553525

(51) Int. Cl. A61K 31/74; A61K 38/02; A61P 17/00; A61K 38/39; A61K 31/315; A61K 31/28; A61K 31/785; A61K 31/78; A61Q 19/00

(52) U.S. Cl. 424/78.03; 514/2; 514/12; 514/494; 514/492; 424/78.02

(57) ABSTRACT

The invention relates in particular to a composition for topical application to the skin, comprising, in a physiologically acceptable medium, at least one peptide increasing the expression of mechanoreceptors in the cells of the skin and at least one tensing agent. According to one alternative the agent increasing the expression of mechanoreceptors in the cells of the skin is selected from zinc salts, copper salts, manganese salts, derivatives thereof and mixtures thereof. The invention likewise relates to a skincare kit comprising at least two compositions comprising respectively the tensing agent and the agent increasing the expression of mechanoreceptors in the cells of the skin. The invention also relates to a method of cosmetic treatment of the skin that comprises simultaneous or sequential application of said compositions.

The present invention relates to the field of skincare and is directed in particular to improving the appearance of the skin and/or the complexion.

The invention relates in particular to a composition for topical application to the skin, comprising, in a physiologically acceptable medium, at least one peptide increasing the expression of mechanoreceptors in the cells of the skin and at least one tensing agent.

In particular the peptides used according to the invention are peptides of matrix proteins selected in particular from collagen, fibronectin and laminin.

According to one alternative the agent increasing the expression of mechanoreceptors in the cells of the skin is selected from zinc salts, copper salts, manganese salts, derivatives thereof and mixtures thereof.

The invention also relates to a skincare kit comprising at least two compositions comprising respectively the tensing agent and the agent increasing the expression of mechanoreceptors in the cells of the skin.

The invention also relates to a method of cosmetic treatment of the skin which comprises the simultaneous or sequential application of said compositions.

The invention also relates to the cosmetic use, in a composition comprising a physiologically acceptable medium, of at least one agent increasing the expression of mechanoreceptors in the cells of the skin in combination with a tensing agent, or to the use of a composition containing said combination, for promoting the improvement of the homeostasis of the skin, the increase of the thickness of the skin, the improvement of the radiance of the complexion, the density of the skin, the regeneration and/or the reorganization of the papillary dermis, the regeneration and/or the reorganization of the extracellular matrix and/or the improvement of the firmness, elasticity and/or tonicity of the skin.

The invention is directed in particular to the treatment of the skin of the face and/or neck. However, the compositions of the invention can also be applied to areas of the body exhibiting a loss of elasticity and/or firmness, such as the stomach and the thighs.

The skin constitutes a physical barrier between the body and its environment. It is composed of two tissues: the epidermis and the dermis.

The epidermis is a keratinizing multi-layered epithelium which undergoes continual renewal. Keratinocytes make up the primary epidermal cell population and are responsible for maintaining the epithelial structure and its barrier function. The epidermis rests on an acellular basal membrane, called the dermoepidermal junction, which ensures cohesion with the dermis.

The epidermis is composed of a number of strata of cells, the deepest of which is the basal stratum, which is composed of undifferentiated cells. Over time these cells will undergo differentiation and will migrate towards the surface of the epidermis, thereby making up the different epidermal strata, until, at the surface of the epidermis, they will form the corneocytes, which are dead cells which are removed by desquamation. This surface loss is compensated by the migration of cells from the basal stratum towards the surface of the epidermis. The process is one of continuous renewal of the skin.

The dermis is an elastic and compressible conjunctive support tissue of mesodermal origin and consists primarily of fibroblasts and an extracellular matrix which is composed of fibrous proteins (collagens and elastin) and non-fibrous proteins (proteoglycans and glycoproteins). The dermis is a feeder tissue for the epidermis, but also plays a fundamental part in the development and growth of the epidermis, and also in its differentiation. The fibroblasts and the extracellular matrix also influence the mechanical properties of the skin, particularly its elasticity, tonicity and firmness. The fibroblasts and the extracellular matrix also influence the density of the skin.

The homeostasis of the skin, and in particular of the epidermis, results from a finely regulated balance between the processes of proliferation and differentiation of the cells of the skin. These proliferation and differentiation processes are perfectly regulated: they participate in the renewal and/or regeneration of the skin and lead to the maintenance of a constant skin thickness, and in particular a constant epidermal thickness. This homeostasis of the skin is also involved in maintaining the mechanical properties of the skin.

However, this homeostasis of the skin can be affected by certain physiological factors (age, menopause, hormones, etc.) or environmental factors (UV stress, pollution, oxidizing stress, irritant stress, etc.). The regenerative potential of the epidermis becomes less great: the cells of the basal layer divide less actively, which leads in particular to a slowdown and/or decrease in epidermal renewal. Consequently, cellular renewal no longer compensates for the loss of the cells removed at the surface, leading to atrophy of the epidermis and/or to a decrease in the thickness of the skin and/or a loss of elasticity and/or tonicity and/or firmness of the skin and/or the formation of wrinkles or fine lines.

These clinical signs are visible on the skin of the face and/or neck, but also on the skin of the body, particularly in areas which exhibit a loss of firmness and/or elasticity, such as the stomach and/or thighs.

The alterations in epidermal homeostasis are also manifested in a dull and/or poorly defined appearance to the complexion of the skin.

This phenomenon may be accentuated by the menopause: women complain of their skin tightening and becoming dry, or even of the appearance of xerosis. The hormonal deficits associated with the menopause are accompanied in particular by a drop in metabolic activity, which can result in a decrease in the proliferation of the keratinocytes and in an increase in epidermal differentiation.

The need is understood, therefore, to have agents capable of promoting the homeostasis of the skin in order to maintain and/or increase the thickness of the skin, particularly the skin of the face and/or neck, and thus to maintain and/or improve the mechanical properties of the skin, particularly the skin of the face and/or neck, and/or to promote radiance in the complexion.

The prior art discloses the use of soluble cosmetic agents for promoting cellular renewal. They include, for example, retinoic acid derivatives, and especially retinol, also known as vitamin A, and esterified derivatives of retinol, which have the effect of promoting proliferation of the keratinocytes and of inhibiting their differentiation, thereby making it possible to stimulate epidermal renewal, to increase the thickness of the epidermis and/or to promote radiance in the complexion.

The soluble cosmetic agents act classically via a bond to a receptor which initiates intracellular responses, leading to a regulation of the expression of proteins which are involved in the processes of epidermal proliferation and/or differentiation. This is termed a direct ‘biological’ effect.

The Applicant has now shown, surprisingly and unexpectedly, that an improvement in the homeostasis of the skin and/or in the radiance of the complexion can be obtained via a biomechanical effect provided by the topical application of an effective amount of cosmetic agents, especially tensing agents.

The Applicant has shown in effect that the topical application of an effective amount of tensing agents, such as acrylic copolymers, to a model of reconstructed skin had the effect of modulating the expression of proteins involved in the homeostasis of the skin.

By ‘biomechanical effect’ according to the invention is meant the capacity of a cosmetic agent, and in particular of a tensing agent, to induce a biological response in the cells of the epidermis and/or dermis, via a mechanical effect which is effective at the surface of the skin (stratum corneum).

By ‘mechanical effect which is effective at the surface of the skin’ is meant the capacity of a cosmetic agent to induce biologically effective mechanical tensions, i.e. mechanical tensions capable of transmitting a mechanical perturbation from cell to cell or via the extracellular matrix, and involving the activation of mechanoreceptors which are present on the membranes of said cells. These cells are referred to as ‘biologically sensitive to mechanical tensions’: interest attaches in particular to the cells of the epidermis and dermis, and especially to the keratinocytes and fibroblasts.

These mechanical tensions, in contrast to conventional stimulation by soluble molecules of the kind used hitherto, have the effect of modifying, via membrane receptors or ‘mechanoreceptors’, an equilibrium which is established between the extracellular matrix and a cell, or between two adjacent cells.

The mechanical tensions are transmitted in the cell in the form of biochemical signals via membrane receptors or mechanoreceptors.

These mechanoreceptors are membrane receptors which are sensitive to mechanical tensions, in other words membrane receptors capable of inducing an intracellular biological response in response to a mechanical perturbation. They include the integrins (Pommerenke et al., Eur J Cell Biol 1996 June; 70(2): 157-64), PECAM1 receptors (Fujiwara et al., Cell struct funct 2001 February; 26(1): 11-17) or else PDGF growth factor receptors (Li et al., Cell Signal 2000 July; 12(7): 435-45).

The tensions, by inducing mechanical perturbation of these receptors, in a first step trigger activation of multiple second messengers. The tensions activate, in particular, protein tyrosine kinase (PTK), protein kinase C(PKC), the G proteins rac and cdc42, or induce the release of calcium flows. The activation of these various signalling pathways leads to the activation of protein kinases from a single family, the MAPkinases, Erk1, Erk2 and p38. The MAPKs, once activated, induce the activation of specific transcriptional factors which regulate the expression of numerous genes involved in the homeostasis of keratinocytes. These activation mechanisms are, moreover, well regulated: in the course of tensions, in particular, Erk induces the expression of MAPK phosphatases, which are known to inhibit Erk. This process allows the cells to control the signals induced by the tensions, and to prevent pathological hyperproliferation of the keratinocytes.

To the knowledge of the Applicant there has never to date been any description or suggestion of improving epidermal homeostasis and/or the radiance of the complexion by the topical application to the skin of an effective amount of a cosmetic agent having a biomechanical effect, in particular a tensing agent.

The prior art has disclosed the use of tensing agents to obtain a superficial and immediate visual effect of smoothing of the microrelief of the skin, particularly smoothing of wrinkles and fine lines. These agents are described as being capable of forming a film which causes the retraction of the stratum corneum, which is the surface, horny layer of the epidermis. The cosmetic or dermatological use of such polymer systems for attenuating the alterations in the microrelief of the skin that are associated with age is described in patent application WO 98/29091. Other tensing agents consist of dispersions of inorganic colloidal particles, particularly silica, as described in patent applications FR-A-2 823 113, FR-2 843 024 and FR-2 659 551 or in U.S. Pat. No. 3,819,825 and U.S. Pat. No. 4,777,041, for example. Further tensing agents are mixed silicates such as those described in patent application FR-2 816 315.

To the knowledge of the Applicant, however, there has never to date been any description of a biological effect of the tensors, and in particular of their capacity to regulate the expression of proteins involved in the homeostasis of the skin, or of their use to promote the homeostasis of the skin, in particular to improve the thickness of the skin and/or to improve the radiance of the complexion and/or to improve the mechanical properties of the skin, and/or to improve the density of the skin, and/or to promote regeneration and/or reorganization of the papillary dermis, and/or to promote regeneration and/or reorganization of the extracellular matrix, in particular at the level of the face and/or neck, but also at the level of certain areas of the body (e.g. stomach, thighs).

This is the context in which the Applicant conceived of using, in combination with these tensing agents, agents which induce and/or increase the expression and therefore the number of mechanoreceptors in the cells of the skin in order to increase the capacity of said cells to respond to mechanical stresses, and thereby to potentialize and/or increase and/or prolong the biological response induced by these tensing agents. This combination is advantageous, moreover, in that it makes it possible to limit the effective amount of tensing agents needed to obtain the desired biological effect, thereby making it possible to optimize the comfort of the cosmetic compositions containing them.

By ‘mechanoreceptors’ according to the invention are meant, in particular, membrane receptors which are sensitive to mechanical tensions, in other words membrane receptors which are capable of inducing an intracellular biological response in response to a mechanical perturbation.

They include the integrins (Pommerenke et al., Eur J Cell Biol 1996 June; 70(2): 157-64), PECAM1 receptors (Fujiwara et al., Cell struct funct 2001 February; 26(1): 11-17) or else PDGF growth factor receptors (Li et al., Cell Signal 2000 July; 12(7): 435-45).

Particular interest will be attached to the group of the integrins, and especially to the class of β1 integrins which are involved in the sensitivity of the cells to mechanical stresses.

Integrins are adhesion molecules which are involved in cell-cell and cell-matrix interactions. They are heterodimeric receptors composed of two subunits, α and β, which are associated non-covalently. More than 17 chains of subunit α and 8 chains of subunit β have been described, which associate to form 23 different heterodimers.

The transmembrane region of the α subunits is composed of an α helix, very highly conserved from one subunit to another, which is responsible for the function of anchoring the integrin to the membrane, and which participates in signal transduction.

The cytoplasmic region of the β subunits, which is very highly conserved from one subunit to another, is responsible on the one hand for the formation of the heterodimer and on the other hand for bonding with structural proteins of the cytoskeleton; this combination also regulates signal transduction.

The heterodimers of integrins can be classed according to their substrate; it is known in particular that:
heterodimers α1β1 and α2β1 bind to collagen;
heterodimers α4β1, α5β1, α8β1 and αvβ1 bind to fibronectin;
heterodimers α1β1, α2β1, α3β1 and α6β1 bind to laminins.

Collagen, fibronectin and laminins are matrix proteins or proteins of the extracellular matrix which participate in the adhesion of the cells and which play an important part in migration and in cell signalling. In the course of the processes of adhesion and of cell migration, the cells interact with the matrix molecules via membrane receptors and in particular the integrins as described above. This interaction initiates intracellular responses which are involved in cell signalling, cell differentiation, migration and/or cell proliferation.

Peptides mimicking the structure of certain regions of these matrix proteins have been defined in the prior art: described in particular is the use of peptides of fibronectin (WO 03/008438), collagen peptides (WO 03/007905) and peptides of fibronectin (WO 03/077936) in compositions in order to increase cellular adhesion.

The present invention relates to a composition comprising, in a physiologically acceptable medium, at least one agent increasing the expression of mechanoreceptors in the cells of the skin and at least one tensing agent.

By ‘agent increasing the expression of mechanoreceptors in the cells of the skin’ is meant in particular, according to the invention, any agent capable of inducing or of stimulating the expression of mechano-receptors in the cells of the skin, particularly in the cells of the epidermis and the dermis (e.g. keratinocytes, fibroblasts).

Interest attaches preferably to agents which increase the expression of integrins, and particularly to agents which increase the expression of β1 integrins.

Such agents may be selected according to conventional methods of detection by immunofluorescence or by quantitative RT-PCR. Preference will be given to using the quantitative RT-PCR technique.

The principle of detection by immunofluorescence consists in contacting cells in culture with the agents under test and then in visualizing the effect of said agents on the expression of mechanoreceptors and in particular of integrins (e.g. β1 integrins) by using anti-integrin antibodies and secondary antibodies coupled to a fluorescent marker (fluorescein).

The general principle of the quantitative RT-PCR technique, which is preferred according to the invention, comprises, for example, the following steps:
the concentrations of the agents under test are selected on the basis of a cytotoxicity study under the conditions of the assay;
human keratinocytes or fibroblasts are cultivated in a culture medium adapted to these different cell types;
the culture medium is exchanged for the same medium containing or not containing (control) the agent under test at the various concentrations selected;
after 24 h of incubation, for example, the mRNAs are extracted and the traces of DNA are removed by treatment with DNAse, which is subsequently deactivated;
then a reverse-transcription reaction is carried out, followed by quantification, by fluorescence, of the cDNA synthesized;
a first series of Q-PCRs is carried out on the β actin marker (check) in order to verify the homogeneity of the preparations to be compared;
subsequently Q-PCRs are carried out in triplicate using pairs of primers specific for the β-actin sequences, and markers specific for mechanoreceptors and in particular for integrins (e.g. β1 integrins);
next, the differential expression of the integrins is evaluated by fluorescence analysis in amplified DNA;
a selection is made of the agents for which an increase in the fluorescence intensity is obtained, corresponding to an increase in the expression of integrins relative to the control condition (that not treated with the agent).

The PCR reactions (polymerase chain reaction) can be performed in particular by quantitative PCR with the “Light Cycler” system (Roche Molecular Systems Inc.) and in accordance with the procedures recommended by the supplier.

According to one first embodiment the agent increasing the expression of mechanoreceptors in the cells of the skin is a peptide.

The invention accordingly provides a composition for topical application to the skin, comprising, in a physiologically acceptable medium, at least one tensing agent and at least one peptide increasing the expression of mechanoreceptors in the cells of the skin.

This peptide may increase the expression of mechanoreceptors in the cells of the skin in particular by either (i) binding to the target mechanoreceptors or (ii) binding to any other membrane receptor capable of inducing an intracellular response leading to an increase in the expression of the target mechanoreceptors, in particular of integrins.

Examples of peptides which can be used according to the invention include, in particular, mimetic peptides of matrix proteins, their homologues or derivatives, which are capable of binding to the target mechanoreceptors.

In particular the mimetic peptides of matrix proteins will be selectable from mimetic peptides of collagen, fibronectin or laminin.

By ‘mimetic peptides of matrix proteins’ are meant peptide sequences which are contained within the native sequences of matrix proteins that are identified as being the key sequences for the function of cellular adhesion, or peptide sequences which are homologous with sequences contained in the native sequences of matrix proteins that are identified as being the key sequences for ensuring the function of cellular adhesion. These key sequences of the matrix proteins are involved in particular in the binding of the proteins with the mechanoreceptors.

These peptides are capable of mimicking the structure of certain regions of said matrix proteins and thus of inducing the same types of signals as them: in particular, they are capable especially of binding to the target mechanoreceptors and of inducing an increase in the expression of said mechanoreceptors.

These peptides will generally have a sequence ranging from 2 to 25 amino acids, in particular from 4 to 16 amino acids.

The peptides that can be used according to the invention include in particular:

1) fibronectin peptides, their homologues and derivatives, such as the peptides of sequence (AA)ₙ-Leu-Asg-Ala-Pro-(AA)ₙ in which AA is any amino acid or derivative thereof and n is between 0 and 2, and in which the amino acids may be in the L (levogyratory), D (dextrogyratory) or DL form. Peptides of this kind are described in patent application WO 03/008438, which is incorporated by reference in the present invention.

Preference will be given to using the hexapeptide of sequence Lys-Leu-Asp-Ala-Pro-Thr, which is homologous with a sequence contained in the subunit III of fibronectin, a homologue or a derivative of this peptide. This peptide is sold by Vincience under the name VINCI 02®. It stimulates the expression of β1 integrins on cells in culture, as described in patent application WO 03/008438.

2) collagen peptides, their homologues and derivatives, such as the peptides of sequence (Gly-Pro-Gln)ₙ-NH₂ in which n is between 1 and 3, and in which the amino acids may be in the L, D or DL form, as described in patent application WO 03/007905, which is incorporated in the present application by reference.

Preference will be given to using the hexapeptide of sequence Gly-Pro-Gln-Gly-Pro-Gln whose sequence is contained in the sequence of collagen, or a homologue or derivative of this peptide. This peptide is sold by Vincience under the name COLLAXYL®.

It stimulates the expression of P1 integrins when it is applied to skin ex vivo as described in Perrin et al. (Int J Tissue React, 2004; 26(3-4): 97-104).

3) laminin peptides, their homologues and derivatives, such as:
the hexapeptide SERILESINE® sold by Lipotec, whose sequence is homologous with a sequence contained in the α chain of laminin. This peptide is described as increasing the expression of (6 integrins in fibroblasts and keratinocytes in culture;
peptides of sequence X₁-Y-Phe-Thr-X₂-Ala-Thr-Z-Ile-X₃-Leu-X₄-Phe-Leu-X₅ in which X₁, X₂, X₃, X₄ and X₅=Arg, Lys or H is, Y=Asp or Glu, and Z=Asn or Gln, as described in patent application WO 03/077936, incorporated by reference in the present application. In particular, the oligopeptide Arg-Asp-Phe-Thr-Lys-Ala-Thr-Asn-Ile-Arg-Leu-Arg-Phe-Leu-Arg, whose amino acid sequence is homologous with a sequence contained in the sequence of laminin. This peptide stimulates the expression of β1 integrins, as described in patent application WO 03/077936. This peptide is sold by Vincience under the name VINCI 01®.

The invention accordingly provides a composition comprising at least one peptide increasing the expression of mechanoreceptors in the cells of the skin and at least one tensing agent, in which the peptide is selected from:
a) a fibronectin mimetic peptide of sequence (AA)ₙ-Leu-Asg-Ala-Pro-(AA)ₙ in which AA is any amino acid or derivative thereof and n is between 0 and 2;
b) a collagen mimetic peptide of sequence (Gly-Pro-Gln)ₙ-NH₂ in which n is between 1 and 3;
c) a laminin mimetic peptide of sequence X₁-Y-Phe-Thr-X₂-Ala-Thr-Z-Ile-X₃-Leu-X₄-Phe-Leu-X₅ in which X₁, X₂, X₃, X₄ and X₅=Arg, Lys or H is, Y=Asp or Glu and Z=Asn or Gln;
and derivatives thereof.
   In particular the peptide is selected from:
d) a fibronectin mimetic hexapeptide of sequence Lys-Leu-Asp-Ala-Pro-Thr;
e) a collagen mimetic hexapeptide of sequence Gly-Pro-Gln-Gly-Pro-Gln;
f) a laminin mimetic oligopeptide of sequence Arg-Asp-Phe-Thr-Lys-Ala-Thr-Asn-Ile-Arg-Leu-Arg-Phe-Leu-Arg;
homologues thereof and derivatives thereof.

These peptides may be of natural or synthetic origin.

By ‘natural origin’ is meant a peptide in the pure state or in solution at different concentrations which is obtained by various processes of extraction from a keratinic material (skin, nail, hair, especially hair) of natural origin or from conjunctive tissues.

By ‘synthetic origin’ is meant a peptide in the pure state or in solution at different concentrations which is obtained chemically or by production in an organism following introduction into said organism of the elements necessary for said production.

These peptides may be obtained by chemical or enzymatic synthesis from the constituent amino acids or their derivatives, or by managed hydrolysis of natural (vegetable or animal) proteins or else by biotechnology in accordance with conventional techniques.

By ‘homologue of these peptides’ is meant, in particular, any peptide sequence identical to at least 50%, preferably to at least 80% and more preferably to at least 95% of said peptide sequences identified above, in the same species or in a different species; in the latter case, it is also denoted by ‘orthologous polypeptide’.

Also, ‘percentage identity’ between two peptide sequences or amino acid sequences is intended to denote a percentage of amino acid residues which are identical between the two sequences under comparison that is obtained after the best alignment, i.e. the optimum alignment achieved, for example, using the Smith-Waterman local homology algorithm (1981, Ad. App. Math. 2: 482), using the Neddleman-Wunsch local homology algorithm (1970, J. Mol. Biol. 48: 443), using the Pearson-Lipman similarity search method (1988, Proc. Natl. Acad. Sci. USA 85: 2444), or using computer software that utilizes these algorithms (GAP, BESTFIT, BLAST P, BLAST N available on the site NCBI, FASTA and TFASTA in the Wisconsin Genetics Software Package, Genetics Computer Group, 575 Science Dr, Madison, Wis.).

By ‘derivative’ of these peptides is meant, in particular, a peptide modified by acylation on its N-terminal function and/or by esterification on its C-terminal function.

The peptides may be solubilized beforehand in one or more cosmetically acceptable solvents such as water, propylene glycol, butylene glycol, ethoxylated or propoxylated diglycols, ethanol, propanol or isopropanol.

They may alternatively be solubilized in a cosmetic vector such as liposomes, or adsorbed on organic or inorganic supports.

These peptides are used in the composition according to the invention in an amount effective for increasing the expression of integrins.

The amount of peptides which can be used according to the invention may range from 0.01% to 20% by weight relative to the total weight of the composition, preferably from 0.1% to 10% by weight relative to the total weight of the composition.

The invention likewise relates to a composition for topical application to the skin, comprising, in a physiologically acceptable medium, at least one tensing agent and at least one agent increasing the expression of mechanoreceptors in the cells of the skin, characterized in that:
when the agent increasing the expression of mechanoreceptors is a zinc gluconate, the tensing agent is not a colloidal particle;
when the agent increasing the expression of mechanoreceptors is a manganese gluconate, the tensing agent is not selected from a graft polymer or a mixed silicate.

According to one particular embodiment of the invention the agent increasing the expression of mechanoreceptors and in particular of integrins is selected from zinc salts, manganese salts, copper salts, derivatives thereof and mixtures thereof.

By ‘salts’ are meant organic or inorganic salts.

Possible organic salts include gluconate, carbonate, acetate, citrate, oleate or oxalate.

Possible inorganic salts include mineral salts such as chloride, borate, nitrate, phosphate or sulphate.

Alternatively it will be possible to use zinc, copper and manganese in an ionic form, in the form of salts or in the form of natural extracts, plant extracts or extracts from microorganisms, particularly bacterial extracts, which are rich in zinc, copper and manganese.

Use will be made in particular of organic salts of zinc, of copper of or manganese, derivatives thereof or mixtures thereof.

Preference will be given to using at least one gluconate salt selected from a zinc gluconate, a copper gluconate, derivatives thereof and mixtures thereof.

These zinc, copper and manganese gluconates have been described as being capable of increasing the expression of integrins, in particular the α2, α3, α6, αv and β1 integrins, on keratinocytes in culture or in reconstructed skins (Tenaud et al., British Journal of Dermatology, 1999: 140; 26-34).

These gluconate salts are sold in particular by Labcatal.

The zinc salts, copper salts or manganese salts are present in the composition in an amount effective for increasing the expression of integrins. It will be possible to use an amount ranging from 0.01% to 20% by weight relative to the total weight of the composition, preferably from 0.1% to 10% by weight relative to the total weight of the composition.

By ‘derivatives’ are meant salts which are complexed with sugars or with amino acids.

According to one particular embodiment the salts will be selected from zinc salts, manganese salts and mixtures thereof.

By “tensing agent” that can be used in accordance with the invention is meant a compound liable to have a tensing effect, in other words able to stretch the skin.

Generally speaking, a tensing agent, according to the invention, refers to any compounds which are soluble or dispersible in water at a temperature ranging from 25° C. to 50° C. at a concentration of 7% by weight in water or at the maximum concentration at which they form a medium of homogeneous appearance, and which at this concentration of 7% or at this maximum concentration in water produce a retraction of more than 15% in the test described below.

The maximum concentration at which they form a medium having a homogeneous appearance is determined to ±10% and preferably to ±5%.

By ‘medium having a homogeneous appearance’ is meant a medium which does not exhibit aggregates visible to the naked eye.

To determine said maximum concentration, the tensing agent is added gradually to water, with stirring using a deflocculator device, at a temperature ranging from 25° C. to 50° C., and then the mixture is kept with stirring for an hour. Inspection then takes place after 24 hours to determine whether the mixture thus prepared has a homogeneous appearance (absence of aggregates visible to the naked eye).

The tensing effect may be characterized by an in vitro retraction test.

Beforehand, and as described above, a homogeneous mixture of the tensing agent is prepared in water, at the concentration of 7% by weight or at the maximum concentration defined above.

30 μl of the homogeneous mixture are placed on a rectangular test specimen (10×40 mm, hence having an initial width W₀ of 10 mm) of elastomer having a modulus of elasticity of 20 MPa and a thickness of 100 μm.

After 3 h of drying at 22±3° C. and 40±10% relative humidity (RH), the elastomer test specimen exhibits a retracted width, recorded as W₃ₕ, owing to the tension exerted by the applied tensing agent.

The tensing effect (TE) of said agent is then quantified as follows:

‘TE’=(W₀−W₃ₕ/W₀)×100 in %

where W₀=initial width, 10 mm
and W₃ₕ=width after 3 h of drying

The tensing agent may be selected from:
a) animal or vegetable proteins and their hydrolysates;
b) polysaccharides of natural origin;
c) mixed silicates;
d) colloidal particles of inorganic fillers;
e) synthetic polymers;
   and mixture of these.

A person skilled in the art will know how the select, from the chemical categories listed above, the materials conforming to the tensing test as described above.

These various categories of tensing agents will now be described.

a) Vegetable Proteins and their Hydrolysates

Examples of vegetable proteins and vegetable protein hydrolysates which can be used as tensing agents in accordance with the invention comprise proteins and protein hydrolysates of maize, rye, wheat, buckwheat, sesame, spelt, tobacco, pea, bean, lentil, soya, almond and lupin.

Animal proteins which can be used in accordance with the invention include, in particular, the proteins extracted from silk, milk, whey and egg.

### b) Polysaccharides of Natural Origin

The polysaccharides of natural origin which are suitable for formulating compositions in accordance with the invention are any polysaccharides of natural origin that are capable of forming gels of either thermoreversible or crosslinked type.

Preference will be given to using polysaccharides capable of forming thermoreversible gels. By thermoreversible is meant that the gel state of these polymer solutions is obtained reversibly once the solution has been cooled below the gelling temperature, which is a characteristic of the polysaccharide used.

A first class of polysaccharides of natural origin which can be used in the present invention is composed of the carrageenans and more particularly of kappa-carrageenan and iota-carrageenan. These are linear polysaccharides which are present in certain red algae. They are composed of alternating β-1,3 and α-1,4 galactose residues, it being possible for many galactose residues to be sulphated. This class of polysaccharides is described in chapter 3 of the book “Food Gels” edited by Peter HARRIS, Elsevier 1989.

Another class of polysaccharides which can be used is composed of the agars. These are also polymers extracted from red algae, and are composed of alternating 1,4-L-galactose and 1,3-D-galactose residues. This class of polysaccharides is also described in chapter 1 of the book “Food Gels” mentioned above.

A third class of polysaccharides is composed of polysaccharides of bacterial origin, which are called gellans. These are polysaccharides composed of an alternation of glucose, glucuronic acid and rhamnose residues. These gellans are described in particular in chapter 6 of the book “Food Gels” mentioned above. Finally, in the case of polysaccharides forming gels of crosslinked type, induced in particular by addition of salts, mention will be made of the polysaccharides belonging to the class of the alginates and the pectins.

These tensing polysaccharides may be present in the form of microgels as described in patent application FR 2 829 025 or not.

It is also possible to make mention of tensing systems comprising:
a polysaccharide and a polyhydroxylated moisturizing agent, such as the system described in patent application FR 2 828 810;
a polysaccharide of pullulan type, as described in U.S. Pat. No. 6,703,027;
a polysaccharide of Biopolymer B16 type, as described in U.S. Pat. No. 5,175,279.

### c) Mixed Silicates

Another class of tensing agents which can be used in accordance with the invention is composed of mixed silicates. By this expression are meant all of the silicates of natural or synthetic origin which contain at least two different cations selected from alkali metals (for example Na, Li, K) or alkaline-earth metals (for example Be, Mg, Ca) and transition metals.

Preference is given to using phyllosilicates, namely silicates having a structure in which the SiO₄ tetra-hedra are organized in sheets between which the metal cations are enclosed.

One class of silicates that is particularly preferred as tensing agents is that of the laponites. Laponites are magnesium lithium sodium silicates which have a layer structure similar to that of montmorillonites. Laponite is the synthetic form of the natural mineral known as hectorite. Use may be made, for example, of the laponite sold under the name Laponite XLS or Laponite XLG by Rockwood.

### d) Colloidal Particles of Mineral Filler

By “colloidal particles” are meant particles in dispersion in an aqueous, aqueous-alcoholic or alcoholic medium, preferably an aqueous medium, which have a number-average diameter of between 0.1 and 100 nm, preferably between 3 and 30 nm.

The colloidal particles according to the invention have no thickening property in water, alcohol, oil and all other solvents. At a concentration greater than or equal to 15% by weight in water, the viscosity of the solutions thus obtained is less than 0.05 Pa·s for a shear rate of 10 s⁻¹. The measurements are made at 25° C. using a Haake RheoStress RS150 rheometer in cone/plate configuration, the measurements of the measuring cone being as follows: 60 mm diameter and 2° angle.

These particles are generally prepared by a sol-gel process and are therefore different in particular from particles of fumed silica, which undergo agglomeration in water to form larger aggregates.

The colloidal particles of mineral filler which can be used in accordance with the invention are generally selected from colloidal particles of silica, cerium oxide, zirconium oxide, alumina, calcium carbonate, barium sulphate, calcium sulphate, zinc oxide and titanium dioxide, colloidal particles of platinum, mixed colloidal particles such as, for example, titanium dioxides with one or more coatings, such as titanium dioxide with a silica coating. In the composition according to the invention preference will be given to using colloidal silicas or colloidal silica-alumina composite particles.

### Colloidal Particles of Silica

By colloidal silicas are meant, for the purposes of this application, colloidal particles of silica in dispersion in an aqueous, aqueous-alcoholic or alcoholic medium. The colloidal particles of silica have a diameter ranging from 0.1 to 100 nm and preferably from 3 to 30 nm. These particles are present in the form of aqueous dispersions and do not have any thickening property in water, alcohol, oil and all other solvents. At a concentration greater than or equal to 15% by weight in water, the viscosity of the solutions thus obtained is less than 0.05 Pa·s for a shear rate of 10 s⁻¹. The measurements are made at 25° C. using a Haake RheoStress RS150 rheometer in cone/plate configuration, the measurements of the measuring cone being as follows: 60 mm diameter and 2° angle.

Colloidal silicas which can be used in the composition according to the invention include, for example, those sold by Catalysts & Chemicals under the names Cosmo S-40 and Cosmo S-50.

### Colloidal Silica-Alumina Composite Particles

The colloidal particles of mineral fillers which can be used according to the invention may also be selected from colloidal silica-alumina composite particles. By silica-alumina composite is meant particles of silica in which atoms of aluminium have been partly substituted for atoms of silica. By colloidal particles are meant, for the purposes of this application, colloidal particles in dispersion in an aqueous, aqueous-alcoholic or alcoholic medium. The colloidal silica-alumina composite particles have a diameter ranging from 0.1 to 100 nm and preferably from 3 to 30 nm. These particles are present in the form of aqueous dispersions and do not have any thickening property in water, alcohol, oil and all other solvents. At a concentration greater than or equal to 15% by weight in water, the viscosity of the solutions thus obtained is less than 0.05 Pa·s for a shear rate of 10 s⁻¹. The measurements are made at 25° C. using a Haake RheoStress RS150 rheometer in cone/plate configuration, the measurements of the measuring cone being as follows: 60 mm diameter and 2° angle.

At a pH of 7, the colloidal silica-alumina composite particles according to the invention have a zeta potential of less than −20 mV and preferably less than −25 mV. The measurements are made at 25° C. using a Coulter Scientific Instrument DELSA 440SX apparatus.

Colloidal silica-alumina composite particles which can be used in the compositions according to the invention include, for example, those sold by Grace under the names Ludox AM, Ludox AM-X 6021, Ludox HSA and Ludox TMA.

### e) Synthetic Polymers

The synthetic polymers used according to the invention may be in solution or in suspension in a polar or apolar (latex) liquid, particularly in aqueous solution or aqueous dispersion, or in a dry form which is redispersible in a cosmetic solvent.

The synthetic polymers which can be used as a tensing agent may be selected from:
polycondensates, especially polyurethanes;
acrylic polymers;
graft silicone polymers;
water-soluble or water-dispersible polymers containing water-soluble or water-dispersible units and LCST units.

The synthetic polymers according to the invention may in particular be selected from interpenetrating polymer networks (IPNs).

These polymers may take the form, in particular, of random linear copolymers, of interpenetrating polymer networks (IPNs), of polycondensates, of graft silicone polymer and of block polymer. Irrespective of its nature, the synthetic polymeric tensing agent may have a weight-average mass Mw ranging from 3000 to 1 000 000 Da.

### Random Linear Copolymers

The random linear copolymers which are tensors in the sense of the present invention are selected from random copolymers having a linear ethylenic main chain with a molecular weight of less than 600 000 Da (g/mol), preferably a weight-average molecular weight of between 15 000 and 600 000 g/mol, and contain at least 70% of a monomer with a glass transition temperature, Tg, greater than 40° C. (preferably >60° C.) whose corresponding homopolymer is insoluble in water at 25° C., and at least one ionic hydrophilic monomer. This copolymer may also contain a non-majority monomer with a Tg less than 40° C.

These copolymers generally exhibit an overall glass transition temperature greater than or equal to 45° C.

Preference is given to all copolymers composed of:
70% to 90% by weight of at least one aryl or alkyl acrylate and (or) at least one aryl or alkyl methacrylate cited in the list below and (or) styrene
10% to 30% by weight of (meth)acrylic acid.

List of preferred alkyl(meth)acrylates: benzyl acrylate, cyclohexyl acrylate, tert-butyl acrylate, isobornyl acrylate and norbornyl acrylate, methyl, ethyl, isobutyl, cyclohexyl, benzyl, tert-butyl, isobornyl and norbornyl methacrylate, preferably methyl methacrylate and cyclohexyl methacrylate.

Among the abovementioned polymers preference will be given particularly to:
copolymers of methyl methacrylate/methacrylic acid; copolymers of methyl methacrylate/acrylic acid, said copolymers containing between 70% and 90% by weight of methyl methacrylate;
copolymers of ethyl methacrylate/methacrylic acid; copolymers of ethyl methacrylate/acrylic acid, said copolymers containing between 70% and 90% by weight of ethyl methacrylate;
copolymers of isobutyl methacrylate/methacrylic acid; copolymers of isobutyl methacrylate/acrylic acid, said copolymers containing between 70% and 90% by weight of isobutyl methacrylate;
copolymers of benzyl methacrylate/methacrylic acid; copolymers of benzyl methacrylate/acrylic acid, said copolymers containing between 70% and 90% by weight of benzyl methacrylate;
copolymers of benzyl acrylate/methacrylic acid; copolymers of benzyl acrylate/acrylic acid, said copolymers containing between 70% and 90% by weight of benzyl acrylate;
copolymers of cyclohexyl methacrylate/methacrylic acid; copolymers of cyclohexyl methacrylate/acrylic acid, said copolymers containing between 70% and 90% by weight of cyclohexyl methacrylate;
copolymers of cyclohexyl acrylate/methacrylic acid; copolymers of cyclohexyl acrylate/acrylic acid, said copolymers containing between 70% and 90% by weight of cyclohexyl acrylate;
copolymers of tert-butyl methacrylate/methacrylic acid; copolymers of tert-butyl methacrylate/acrylic acid, said copolymers containing between 70% and 90% by weight of tert-butyl methacrylate;
copolymers of tert-butyl acrylate/methacrylic acid; copolymers of tert-butyl acrylate/acrylic acid, said copolymers containing between 70% and 90% by weight of tert-butyl acrylate;
copolymers of isobornyl methacrylate/methacrylic acid; copolymers of isobornyl methacrylate/acrylic acid, said copolymers containing between 70% and 90% by weight of isobornyl methacrylate;
copolymers of isobornyl acrylate/methacrylic acid; copolymers of isobornyl acrylate/acrylic acid, said copolymers containing between 70% and 90% by weight of isobornyl acrylate;
copolymers of norbornyl methacrylate/methacrylic acid; copolymers of norbornyl methacrylate/acrylic acid, said copolymers containing between 70% and 90% by weight of norbornyl methacrylate;
copolymers of norbornyl acrylate/methacrylic acid; copolymers of norbornyl acrylate/acrylic acid, said copolymers containing between 70% and 90% by weight of norbornyl acrylate; and
copolymers of styrene/methacrylic acid; copolymers of styrene/acrylic acid, said copolymers containing between 70% and 90% by weight of styrene.

The copolymers in accordance with the present invention are in the form of a dispersion in a polar liquid. These copolymers are dispersed in water after neutralization with a base.

A preferred copolymer according to the invention is selected from copolymers of methyl methacrylate/methacrylic acid, said copolymers containing between 70% and 90% by weight of methyl methacrylate.

### Interpenetrating Polymers or IPNs

An “interpenetrating polymer network” for the purposes of the present invention means a mixture of two inter-meshed polymers obtained by simultaneous polymerization and/or crosslinking of two types of monomers, the resulting mixture having a single glass transition temperature. Examples of IPNs that are suitable for employment in the present invention, and the process for preparing them, are described in patents U.S. Pat. No. 6,139,322 and U.S. Pat. No. 6,465,001, for example. Preferably the IPN according to the invention comprises at least one acrylic polymer and, more preferably, it further comprises at least one polyurethane or a copolymer of vinylidene fluoride and hexafluoropropylene. According to one preferred embodiment the IPN according to the invention comprises a polyurethane and an acrylic polymer. IPNs of this kind are in particular those of the Hybridur series which are available commercially from Air Products. One particularly preferred IPN is in the form of an aqueous dispersion of particles having a weight-average size of between 90 and 110 nm and a number-average size of approximately 80 nm. This IPN preferably has a glass transition temperature, Tg, which ranges from approximately −60° C. to +100° C. An IPN of this type is sold in particular by Air Products under the trade name Hybridur 875. Other IPNs suitable for use in the present invention are referenced Hybridur X01602 and Hybridur 580.

Other IPNs suitable for employment in the present invention include IPNs composed of a mixture of a polyurethane with a copolymer of vinylidene fluoride and hexafluoropropylene. These IPNs can be prepared in particular as described in patent U.S. Pat. No. 5,349,003. As a variant, they are available commercially in the form of a colloidal dispersion in water, in a ratio of the fluoro copolymer to the acrylic polymer of between 70:30 and 75:25, under the trade names KYNAR RC-10, 147 and KYNAR RC-10, 151 from ATOFINA.

### Polycondensate

According to a second variant the composition may comprise as synthetic polymeric tensing agent at least one polycondensate. Polymers in the form of polycondensates having a tensing effect have been described in particular in patent application WO 98/29092.

Polycondensates include polyurethanes, especially anionic, cationic, nonionic or amphoteric polyurethanes, polyurethane-acrylics, polyurethane-polyvinylpyrrolidones, polyester-polyurethanes, polyether-polyurethanes, polyureas, and mixtures thereof.

The polyurethane may be, for example, a polyurethane, polyurea/urethane or polyurea copolymer which is aliphatic, cycloaliphatic or aromatic and comprises, alone or in a mixture,
at least one sequence originating from a linear or branched aliphatic and/or cycloaliphatic and/or aromatic polyester, and/or
at least one sequence originating from an aliphatic and/or cycloaliphatic and/or aromatic polyether, and/or
at least one sequence containing fluorinated groups.

The polyurethanes may also be obtained from branched or unbranched polyesters or from alkyds containing mobile hydrogens that are modified by reaction with a diisocyanate and a difunctional organic compound (for example a dihydro, diamino or hydroxyamino compound), further containing alternatively a carboxylic acid or carboxylate group, or a sulphonic acid or sulphonate group, or else a neutralizable tertiary amine group or a quaternary ammonium group. Mention may also be made of polyesters, polyester amides, fatty-chain polyesters, polyamides, and epoxy ester resins.

In order to form a polyurethane, mention may be made, as a monomer which carries an anionic group and can be used in polycondensation, of dimethylolpropionic acid, trimellitic acid or a derivative, such as trimellitic anhydride, the sodium salt of the acid 3-sulphopentanediol, or the sodium salt of 5-sulpho-1,3-benzenedicarboxylic acid.

The polycondensates include the polymers sold under the trade names Avalure UR410, Avalure UR405, Avalure UR460 by Noveon, and under the trade names Neorez R974, Neorez R981 and Neorez R970 by Avecia.

Mention may also be made of combinations of polymers, such as polyurethanes having a degree of shrinkage of less than or equal to 20% and acrylic polymers having a degree of shrinkage of less than or equal to 20%, which are described in patent application WO 2005067884.

### Graft Silicone Polymer

Among the synthetic polymeric tensing agents used in the composition according to the invention, mention may be made, as a variant, of graft silicone polymers in particular, as defined in patent application EP-1038519. The polymer in question may more particularly be a polymer comprising a main chain of silicone or polysiloxane (Si—O— polymer) on which is grafted, within said chain and, optionally, at least one of its ends, at least one organic group not containing silicone.

The polymers having a polysiloxane backbone grafted with non-silicone organic monomers according to the invention may be existing commercial products or may be obtained by any means known to a person skilled in the art, in particular by reaction between (i) a starting silicone correctly functionalized on one or more of its silicon atoms and (ii) a non-silicone organic compound which is itself correctly functionalized by a function which is capable of reacting with the functional group or groups carried by said silicone, thereby forming a covalent bond; a classic example of such a reaction is the hydrosilylation reaction between ≡Si—H groups and vinylic groups CH₂═CH—, or else the reaction between thiofunctional groups —SH and the same vinylic groups.

Examples of polymers having a polysiloxane backbone grafted with non-silicone organic monomers that are suitable for an implementation of the present invention, and their particular method of preparation, are described in particular in patent applications EP-A-0582152, WO 93/23009 and WO 95/03776, the teachings of which are included in their entirety in the present description as non-limitative references.

According to one particularly preferred embodiment of the present invention the silicone polymer that is employed, having a polysiloxane backbone grafted with non-silicone organic monomers, is the result of free-radical copolymerization between, on the one hand, at least one non-silicone anionic organic monomer containing ethylenic unsaturation and/or a non-silicone hydrophobic organic monomer containing ethylenic unsaturation and, on the other hand, a silicone having in its chain at least one functional group capable of reacting with said ethylenic unsaturations of said non-silicone monomers, thereby forming a covalent bond, and particularly thio-functional groups.

According to the present invention said ethylenically unsaturated anionic monomers are preferably selected, alone or in mixtures, from linear or branched unsaturated carboxylic acids which optionally are partly or totally neutralized in the form of a salt, it being possible for this or these unsaturated carboxylic acids to be, more particularly, acrylic acid, methacrylic acid, maleic acid, maleic anhydride, itaconic acid, fumaric acid and crotonic acid. The salts that are suitable are, in particular, alkali metal salts, alkaline-earth metal salts and ammonium salts. It will be noted that, likewise, in the final graft silicone polymer, the anionic organic group which constitutes the result of the free-radical (homo)polymerization of at least one anionic monomer of unsaturated carboxylic acid type may, after reaction, be neutralized with a base (sodium hydroxide solution, aqueous ammonia, etc.) in order to bring it into the form of a salt.

According to the present invention the ethylenically unsaturated hydrophobic monomers are preferably selected, alone or in mixtures, from esters of acrylic acid with alkanols and/or esters of methacrylic acid with alkanols. The alkanols are preferably C₁-C₁₈ and more particularly C₁-C₁₂. The preferred monomers are selected from the group consisting of isooctyl (meth)acrylate, isononyl(meth)acrylate, 2-ethylhexyl (meth)acrylate, lauryl(meth)acrylate, isopentyl (meth)acrylate, n-butyl(meth)acrylate, isobutyl (meth)acrylate, methyl(meth)acrylate, tert-butyl (meth)acrylate, tridecyl(meth)acrylate, stearyl (meth)acrylate or mixtures thereof.

One class of silicone polymers having a polysiloxane backbone grafted with non-silicone organic monomers that is particularly suitable for the implementation of the present invention is composed of silicone polymers containing in their structure the unit of formula (I) below:

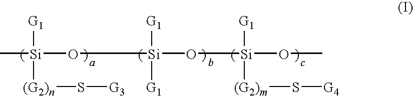

in which the radicals G₁, identical or different, represent hydrogen or a C₁-C₁₀ alkyl radical or else a phenyl radical; the radicals G₂, identical or different, represent a C₁-C₁₀ alkylene group; G₃ represents a polymeric residue resulting from the (homo)polymerization of at least one ethylenically unsaturated anionic monomer; G₄ represents a polymeric residue resulting from the (homo)polymerization of at least one ethylenically unsaturated hydrophobic monomer; m and n are, independently of one another, 0 or 1; a is an integer ranging from 0 to 50; b is an integer which can be between 10 and 350; and c is an integer ranging from 0 to 50, with the proviso that one of parameters a and c is different from 0.

Preferably the unit of formula (I) above has at least one, and more preferably all, of the following features:
the radicals G₁ denote a C₁-C₁₀ alkyl radical;
n is non-zero, and the radicals G₂ represent a divalent C₁-C₃ radical;
G₃ represents a polymeric radical resulting from the (homo)polymerization of at least one ethylenically unsaturated carboxylic acid monomer, preferably acrylic acid and/or methacrylic acid;
G₄ represents a polymeric radical resulting from the (homo)polymerization of at least one C₁-C₁₀ alkyl (meth)acrylate monomer.

Examples of graft silicone polymers conforming to the formula (I) are therefore, in particular, polydimethyl-siloxanes (PDMSs) grafted via a thiopropylene linker with polymeric units of poly(meth)acrylic acid type and/or polyalkyl, especially C₁-C₃, or even C₁, alkyl (meth)acrylate type.

These polymers are referenced under the CTFA name “polysilicone-8”.

One preferred example of a graft silicone polymer is the polysilicone-8 (CTFA name) which is a polydimethyl-siloxane grafted via a thiopropylene linker with mixed polymer units of poly(meth)acrylic acid type and of polyalkyl, especially C₁-C₃, or even C₁, alkyl (meth)acrylate type.

The polymer in question may therefore be a propyl-thio(polymethyl acrylate/methyl methacrylate/methacrylic acid)-grafted polydimethylsiloxane or a propyl-thio(polymethyl acrylate)-, propylthio(polymethyl methacrylate)- and propylthio(polymethacrylic acid)-grafted polydimethylsiloxane. As a variant it may be a propylthio(polyisobutyl methacrylate)- and propyl-thio(polymethacrylic acid)-grafted polydimethyl-siloxane. Preference is given to using a propyl-thio(polymethyl acrylate)/methyl methacrylate/methacrylic acid)-grafted polydimethylsiloxane.

A polymer of this type is available in particular under the trade name VS 80 or VS 70 (at 10% in water) or LO 21 (in powder form) from 3M.

Preferably the number-average molecular mass of the silicone polymers having a polysiloxane backbone grafted with non-silicone organic monomers of the invention varies from 10 000 to 1 000 000 approximately and more preferably from 10 000 to 100 000 approximately.

### Star Polymer

According to yet another possibility, the synthetic polymeric tensing agent which can be used in the composition according to the invention may comprise at least one polymer of “star” structure, represented by the following formula (I):

A-[(M1)p1−(M2)p2 . . . (Mi)pj]n (I)

in which:
A represents a multifunctional centre, of functionality “n”, n being an integer greater than 2, in particular greater than 5;
[(M1)p1−(M2)p2 . . . (Mi)pj] represents a polymeric chain, also called a branch, which is composed of polymerized monomers Mi, which are identical or different, having a polymerization index pj, each branch being identical or different, and being grafted covalently onto said centre A;
i is greater than or equal to 1, and pj is greater than or equal to 2;
   said polymer comprising one or more monomers Mi whose corresponding homopolymer exhibits a Tg of greater than or equal to approximately 10° C., preferably greater than or equal to 15° C., and more preferably greater than or equal to 20° C.; and this or these monomers Mi being present in a minimum amount of approximately 45% by weight, preferably in an amount varying between 55% and 99% by weight, and more preferably between 75% and 90% by weight, relative to the total weight of the entirety of the monomers of the final polymer. These polymers, and the process for preparing them, are described in particular in document EP 1 043 345.

### Block Polymer

As a variant, the synthetic polymeric tensing agents which can be used in the composition according to the invention may be block polystyrene (PS)-polyethyl acrylate (PEA) polymers.

Very generally, a block copolymer is a polymer composed of at least two distinct homopolymers composed solely of monomers A and B respectively. Thus the blocks according to the invention are, respectively, polystyrene (PS) and polyethyl acrylate (PEA) blocks.

In the context of this variant, the polymer may be a triblock polymer of type PS-PEA-PS or else a multiblock polymer of type PS-[PEA-PS]n, or PEA-[PS-PEA]n, where n is a positive integer and is preferably 1.

Advantageously these block polymers are linear copolymers. The molecular weight of this polymer is preferably greater than 10 000 daltons, and more preferably greater than 50 000 daltons. The ratio by weight of the PS and PEA monomers may be defined such that PS/PEA is greater than 1 and preferably such that PS/PEA is greater than 5.

Mention may be made of the triblock polymer PS(30 000)—PEA(10 000)—PS(30 000), which is most particularly suitable for the implementation of the invention. This particularly advantageous block copolymer is a triblock copolymer comprising:
a first block containing units deriving from styrene, having a number-average molecular mass of 30 000 g/mol;
a second block composed of units deriving from ethyl acrylate, having a number-average molecular mass of 10 000 g/mol;
a third block containing units deriving from styrene, having a number-average molecular mass of 30 000 g/mol.

A copolymer conforming to the definition given above may be a copolymer for which the first block and/or the third block and, preferably, the first block and the third block comprise, in addition to the units deriving from styrene, units deriving from methacrylic acid, for example, in a mass ratio (styrene/methacrylic acid) of 98/2.

The synthetic copolymers used according to the invention may also, as a variant, be composed of a random polystyrene polyethyl acrylate copolymer. The ratio by weight of the PS and PEA monomers is defined such that PS/PEA>1 and preferably such that PS/PEA>5.

Alternatively the tensing polymers according to the invention may also be selected from vinyl derivatives such as polyvinyl alcohols and polyvinylpyrrolidones, either in block or else in random form.

Finally, synthetic polymers which are appropriate may be water-soluble or water-dispersible polymers containing water-soluble or water-dispersible units and containing LCST units, said LCST units exhibiting, in particular, a separation temperature in water of 5 to 40° C. at a mass concentration of 1%. This type of polymer is more fully described in patent application FR 2 819 429.

According to one particular embodiment of the invention the tensing agent used in accordance with the invention may be selected from:
vegetable or animal proteins and their hydrolysates;
polysaccharides of natural origin;
synthetic polymers of polycondensate or graft silicone polymer type;
   and mixtures thereof.

Preference will be given to using a tensing agent selected from silicone polymers having a polysiloxane backbone grafted with non-silicone organic monomers.

In particular the silicone polymer comprises in its structure the unit of formula (I) below:

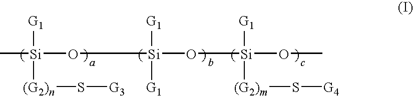

in which the radicals G₁, identical or different, represent hydrogen or a C₁-C₁₀ alkyl radical or else a phenyl radical; the radicals G₂, identical or different, represent a C₁-C₁₀ alkylene group; G₃ represents a polymeric residue resulting from the (homo)polymerization of at least one ethylenically unsaturated anionic monomer; G₄ represents a polymeric residue resulting from the (homo)polymerization of at least one ethylenically unsaturated hydrophobic monomer; m and n are, independently of one another, 0 or 1; a is an integer ranging from 0 to 50; b is an integer which can be between 10 and 350; and c is an integer ranging from 0 to 50, with the proviso that one of parameters a and c is different from 0.

Preferably the unit of formula (I) has at least one, and more preferably all, of the following features:
the radicals G₁ denote a C₁-C₁₀ alkyl radical;
n is non-zero, and the radicals G₂ represent a divalent C₁-C₃ radical;
G₃ represents a polymeric radical resulting from the (homo)polymerization of at least one ethylenically unsaturated carboxylic acid monomer, preferably acrylic acid and/or methacrylic acid;
G₄ represents a polymeric radical resulting from the (homo)polymerization of at least one C₁-C₁₀ alkyl(meth)acrylate monomer.

More particularly still, the graft silicone polymers corresponding to the formula (I) are polydimethyl-siloxanes (PDMSs) grafted via a thiopropylene linker with polymeric units of poly(meth)acrylic acid type and/or of polyalkyl, especially C₁-C₃, or even C₁, alkyl (meth)acrylate type.

A preferred graft silicone polymer is a propyl-thio(polymethyl acrylate/methyl methacrylate/meth-acrylic acid)-grafted polydimethylsiloxane.

According to another preferred embodiment of the invention use will be made of a tensing agent selected from:
(i) mineral tensing agents, such as mixed silicates and colloidal particles of inorganic fillers,
(ii) tensing polymers, especially:
   copolymers of methyl methacrylate/methacrylic acid, said copolymers containing between 70% and 90% by weight of methyl methacrylate;
   interpenetrating polymers comprising a polyurethane and an acrylic polymer,
      and mixtures thereof.

The tensing agents which enter into this preferred embodiment of the invention have the particular feature, when deposited on a glass plate, of forming a mosaic deposit. In the remainder of the description they will be referred to as “mosaic-effect tensing agents”.

A mosaic-effect tensing agent is in accordance with the invention an agent which, when applied to a glass plate, dries to form a tessellated deposit, it being possible for the size and shape of its constituent tessellae to depend on their location relative to the edges of the deposit.

By ‘tessellated deposit’ is meant more precisely a discontinuous deposit made up of a multiplicity of small individualized domains or microdomains.

The tessellae or microdomains are generally small in size. This size may range from 0.1 mm² to several mm².

In particular a mosaic deposit of this kind cannot be peeled or detached from the substrate, in contrast to continuous or semi-continuous deposits which adhere to the substrate and which can be detached or peeled either in a single piece or in two or more relatively large-sized strips.

Moreover, a mosaic deposit of this kind generally exhibits low resistance to water; in other words, in contact with water, the deposit breaks up.

A mosaic deposit of this kind according to the invention is shown in FIG. 1.

The tessellated or mosaic appearance of the deposit results in particular from the fact that the stresses developed by these tensing agents in the course of drying are greater than the forces of cohesion (rigidity) of the deposit.

From a mechanical standpoint, deposits of this kind formed by these mosaic-effect tensing agents may be characterized by their property of fracturing under stresses, which is evaluated for example in the mechanical strength test described below.

In particular the mosaic-effect tensing agents according to the invention form deposits characterized advantageously by a breaking energy of between 0 and 20 J/m² (preferably equal to 0) and a breaking deformation of between 0 and 0.2 mm in the mechanical strength test described below.

Said mechanical strength test consists for example in applying compressive stress to breaking point to the tensing agent at the surface of a flexible and deformable foam.

The tensing agent is deposited at a concentration of 7% by weight in water or at the maximum concentration by weight at which it forms in water at a temperature ranging from 25° C. to 50° C. a medium having a homogeneous appearance.

The use of this foam substrate makes it possible to apply a substantial deformation to the surface-deposited tensing agent, and hence makes it possible to quantify its breaking strength. The mechanical compressive stress is exerted by means of a cylindrical punch 1 mm in diameter, the rate of displacement of the punch being 0.1 mm/s.

The test is carried out using a TA-XT2i texture analyzer sold by Stable Micro System. In this way a curve of force F (in N) as a function of displacement d (in mm) is obtained, from which it is possible to determine the breaking point of the material (tensing agent) or breaking energy W_{break}, expressed in J/m², as shown in FIG. 2.

The breaking energy W_{break} expressed in J/m² corresponds to the area under the curve F=f(d) obtained at the value of the displacement for which a discontinuation F_{break} (N) is observed.

The mosaic-effect tensing agents used in accordance with the invention are advantageous relative to other tensing agents, which form a continuous or semi-continuous deposit which adheres to a flexible substrate such as the skin, in that they allow better distribution, over the whole surface area of the skin, of the tensions exerted, relative to a continuous or semi-continuous deposit. This is because, in the case of a continuous or semi-continuous deposit which adheres to a flexible substrate, the tensions develop in the substrate solely at the periphery of the solid deposit.

In contrast, in the case of a mosaic deposit according to the invention, with the same surface area as the above deposit, the tensions develop at the periphery of each independent tessella of the deposit, thereby considerably increasing the surface area subject to the tensions.

By virtue of these tensions that are more effectively distributed over the entire surface area of the skin on which the deposit is formed, the Applicant has been able to show that the biological effects are obtained much more rapidly: starting from the first few hours after application of said mosaic-effect tensing agent to the skin, and in particular after 48 hours.

Preference will be given to using, as mosaic-effect tensing agent, colloidal particles of silica.

### Other Possible Preferred Mosaic-Effect Tensing Agents Include:

copolymers of methyl methacrylate/methacrylic acid, said copolymers containing between 70% and 90% by weight of methyl methacrylate;
interpenetrating polymers containing a polyurethane and an acrylic polymer.

The tensing agent will be present in the composition in an amount effective for obtaining the desired biological effect according to the invention.

This effective amount will be defined such that the combination of the tensing agent with the agent increasing the expression of mechanoreceptors in the cells makes it possible to obtain the desired biological effect, namely an effect on the homeostasis of the skin.

This effective amount or effective dose can be evaluated, for example, by a DNA array method as described in the illustrative examples below, the general principle of which is as follows:
different doses of tensing agents and agents increasing the expression of mechanoreceptors are applied, in combination, to cells in culture or to a model of epidermis and/or of reconstructed skin;
the mRNAs are extracted from said treated or untreated (control) cells and a ‘reverse’ transcription is carried out using, for example, oligo dT and a P33-labelled deoxynucleotide triphosphate, to give labelled target cDNA sequences;
these target cDNA sequences are hybridized on dedicated minichips containing DNAs specific for the markers that are involved in the physiology of the cells of the skin, and in particular in the homeostasis of the skin (called ‘cDNA probes’);
after washing, the amount of labelled target sequences is measured, and is compared with the control in order to evaluate the variation of expression of the target genes that is induced by the topical application of said tensing agent, relative to the control;
subsequently a selection is made of the amounts or associated effective doses of tensing agents and agents increasing the expression of mechano-receptors for which a variation is obtained in the expression of genes involved in the proliferation (increase) and/or differentiation (decrease) in the cells of the skin relative to an (untreated) control. Advantageously a selection is made of the associated effective doses for which a decrease is obtained in the expression of genes involved in the differentiation of keratinocytes (e.g. corneodesmosin, loricrin, suprabasin) and/or an increase in the genes involved in the regeneration of the skin (e.g. cytokeratins) relative to a control, preferably a variation in the expression by a factor of 2 or more relative to the control.

By way of example the tensing agent may be included in the composition according to the invention in an amount ranging from 0.01% to 30% by weight of active ingredient, preferably from 1% to 30% by weight of active ingredient, relative to the total weight of the composition.

In particular it will be possible to use an effective amount of tensing agent ranging from 2% to 30% by weight, in particular from 3% to 20%, preferably from 4% to 20% by weight of active ingredient relative to the total weight of the composition, for example an amount between 6% and 10% by weight of active ingredient relative to the total weight of the composition.

According to one particular embodiment an effective amount of tensing agent will be used of 3% to 20% by weight of active ingredient relative to the total weight of the composition, preferably of 3% to 7% by weight of active ingredient relative to the total weight of the composition.

By “active ingredient” the intention is to exclude the medium in which the tensing agent is optionally solubilized or in dispersion in its commercial form, as for example in the case of dispersions of colloidal particles.

The composition according to the invention comprises a physiologically acceptable medium, in other words a medium which is compatible with the skin of the face and/or body. It is preferably a cosmetically acceptable medium, in other words a medium which has a colour, odour and feel that are pleasant and which does not give rise to any unacceptable discomfort (stinging, tautness, redness) that might dissuade the consumer from using this composition.

The composition according to the invention may be a bodycare or facial-care composition or a makeup composition.

The composition according to the invention may be in any of the formulated forms conventionally used for topical application, and particularly in the form of dispersions of the aqueous gel or lotion type, emulsions with a liquid or semi-liquid consistency of the milk type, obtained by dispersing a fatty phase in an aqueous phase (O/W) or conversely (W/O), or of suspensions or emulsions with a soft, semi-solid or solid consistency, of cream or gel type, or in the form of a serum or stick, or else of multiple emulsions (W/O/W or O/W/O), of microemulsions, of ionic and/or nonionic vesicular dispersions, or of wax/aqueous phase dispersions. These compositions are prepared in accordance with the customary methods.

Oils which can be used in the composition according to the invention include the following:
hydrocarbon oils of animal origin, such as perhydrosqualene;
hydrocarbon oils of vegetable origin, such as liquid triglycerides of fatty acids containing 4 to 10 carbon atoms, or else, for example, vegetable oils such as apricot kernel oil and shea butter oil;
synthetic esters and ethers, especially those of fatty acids, such as the oils of formulae R¹COOR² and R¹OR² in which R¹ represents the residue of a fatty acid containing 8 to 29 carbon atoms and R² represents a branched or unbranched hydrocarbon chain containing 3 to 30 carbon atoms;
linear or branched hydrocarbons of mineral or synthetic origin, such as volatile or non-volatile liquid paraffins and derivatives thereof, isohexadecane, isododecane, Vaseline, polydecenes, hydrogenated polyisobutene such as Parléam® oil;
natural or synthetic essential oils;
branched fatty alcohols having 8 to 26 carbon atoms, such as octyldodecanol;
partially hydrocarbon-modified and/or silicone-modified fluoro oils like those described in document JP-A-2-295912;
silicone oils such as volatile or non-volatile polydimethylsiloxanes (PDMSs) having a linear or cyclic silicone chain which are liquid or pasty at ambient temperature, especially cyclopolydimethylsiloxanes (cyclomethicones) such as cyclohexasiloxane and cyclo-pentasiloxane; polydimethylsiloxanes containing alkyl, alkoxy or phenyl groups pendantly or at the end of a silicone chain, these groups having 2 to 24 carbon atoms; phenyl silicones such as phenyltrimethicones, phenyldimethicones, phenyltrimethylsiloxydiphenyl-siloxanes, diphenyldimethicones, diphenylmethyl-diphenyltrisiloxanes, 2-phenylethyl trimethylsiloxy-silicates, and polymethylphenylsiloxanes; and
mixtures thereof.

The other fatty substances which may be present in the oily phase are, for example, fatty acids containing 8 to 30 carbon atoms, such as stearic acid, lauric acid, palmitic acid and oleic acid; linear fatty alcohols such as cetyl alcohol and/or stearyl alcohol; pasty fatty substances such as lanolin; waxes; and gums such as silicone gums (dimethiconol).

These fatty substances may be selected variously by the skilled worker in order to prepare a composition having the desired properties of, for example, consistency or texture.

This composition may further comprise various adjuvants commonly used in the field of cosmetology, such as emulsifiers, including esters of fatty acids and of polyethylene glycol, esters of fatty acid and of sorbitan which are optionally polyoxyethylenated, polyoxyethylenated fatty alcohols and the esters or ethers of fatty acid and of sugars such as sucrose or glucose; fillers; preservatives; sequestrants; fragrances; and thickeners and/or gelling agents, such as homopolymers and copolymers of acrylic acid, homopolymers and copolymers of acrylamide and/or of 2-acrylamido-2-methylpropanesulphonic acid (AMPS), modified AMPS (Aristoflex LNC and SNC) products, and xanthan gum.

Fillers include, for example, particles of polyamide (Nylon) in spherical or microfibre form; microspheres of polymethyl methacrylate; ethylene-acrylate copolymer powders; expanded powders such as hollow microspheres and, in particular, the microspheres formed from a terpolymer of vinylidene chloride, acrylonitrile and methacrylate which are sold under the name Expancel; powders of natural organic materials such as powders of starch, particularly of maize starch, wheat starch or rice starch, crosslinked or non-crosslinked, such as powders of starch crosslinked with octenylsuccinic anhydride; silicone resin microbeads such as those sold under the name Tospearl by Toshiba Silicone; silica; metal oxides such as titanium dioxide or zinc oxide; mica; hollow hemispherical particles of silicone such as NLK506 sold by Takemoto Oil and Fat; and mixtures thereof.

The skilled worker will of course take care to select this or these optional additional compounds and/or their amount in such a way that the advantageous properties of the composition according to the invention are not, or not substantially, adversely affected by the intended addition.

The composition according to the invention is applied in accordance with the typical techniques, for example by application of creams, gels, serums or lotions to the skin it is intended to treat, in particular the skin of the body, face and/or neck.

The composition according to the invention may also contain actives having a complementary effect to the combination according to the invention, such as at least one compound selected from desquamating agents, moisturizing agents, agents which stimulate the proliferation and/or differentiation of keratinocytes, agents which stimulate the synthesis of collagen and/or elastin or prevent their breakdown, depigmenting agents, anti-glycation agents, agents which stimulate the synthesis of glycosaminoglycans, antioxidants and free-radical scavengers, and mixtures thereof.

Examples of such actives are as follows: retinol and its derivatives such as retinyl palmitate; ascorbic acid and its derivatives such as magnesium ascorbyl phosphate and ascorbyl glucoside; tocopherol and its derivatives such as tocopheryl acetate; nicotinic acid and its precursors such as nicotinamide; ubiquinone; glutathione and its precursors such as L-2-oxothiazolidine-4-carboxylic acid; plant extracts and especially extracts of rock samphire and of olive leaf; algal extracts and especially laminaria extracts; bacterial extracts; sapogenins such as diosgenin and Dioscorea extracts, in particular wild yam extracts, which contain them; α-hydroxy acids; β-hydroxy acids, such as salicylic acid and n-octanoyl-5-salicylic acid; oligopeptides and pseudodipeptides and their acyl derivatives, especially {2-[acetyl(3-trifluoromethyl-phenyl)amino]-3-methylbutyrylamino}acetic acid and the lipopeptides sold by Sederma under the trade names Matrixyl 500 and Matrixyl 3000; lycopene; and mixtures thereof.

The invention likewise provides a skincare kit comprising at least:
a first composition comprising, in a physiologically acceptable medium, at least one agent increasing the expression of mechano-receptors in the cells of the skin;
a second composition comprising, in a physiologically acceptable medium, at least one tensing agent.

In particular, when the agent increasing the expression of mechanoreceptors in the cells of the skin is a zinc gluconate, the second composition is devoid of organopolysiloxane elastomer.

The agent increasing the expression of mechanoreceptors in the cells of the skin may be selected from (i) peptides, (ii) zinc salts, copper salts or manganese salts, derivatives thereof and mixtures thereof.

Examples of peptides and of zinc salts, copper salts or manganese salts, derivatives thereof and mixtures thereof have been described above in the description.

The tensing agent present in the second composition may be selected from synthetic polymers, animal or vegetable proteins, polysaccharides of vegetable origin in microgel or non-microgel form, mixed silicates, colloidal particles of inorganic fillers and mixtures thereof.

Examples of tensing agents which can be used in accordance with the invention have been described above in the description.

The invention likewise provides a method of cosmetic treatment of the skin which comprises the application, via the use of at least one composition, of at least one agent increasing the expression of mechanoreceptors in the cells of the skin and at least one tensing agent.

The application may be made simultaneously (one composition) or sequentially (two separate compositions).

By “sequential application” is meant successive (immediate) or delayed application.

In particular the agent increasing the expression of mechanoreceptors in the cells of the skin is a peptide.

In particular the method according to the invention is intended especially to promote the homeostasis of the skin and/or improve the mechanical properties of the skin (e.g. firmness, elasticity, tonicity) and/or improve the density of the skin, and/or promote the radiance of the complexion, and/or promote the regeneration and/or reorganization of the papillary dermis, and/or promote the regeneration and/or reorganization of the extracellular matrix, in addition to an immediate effect of smoothing of the skin microrelief and of wrinkles, as provided by the tensing agent.

According to one particular embodiment the method of cosmetic treatment of the skin comprises the sequential application of at least one composition comprising at least one agent increasing the expression of mechano-receptors in the cells of the skin and a composition comprising at least one tensing agent.

For sequential application the composition comprising the agent increasing the expression of mechanoreceptors in the cells of the skin is preferably applied before the composition comprising the tensing agent during the initial days of treatment.

The order in which the compositions are applied is less important after a number of days of treatment.

According to one particular embodiment the composition or compositions according to the invention will be able to be applied to persons exhibiting a dull and/or imprecise complexion, in order to promote the radiance of the complexion.

According to another embodiment the composition or compositions according to the invention will be able to be applied to persons exhibiting a soft and/or flaccid skin or to areas of the body that exhibit a loss of elasticity and/or firmness and/or tonicity.

In particular it will be possible to apply the composition to the face, the stomach and the thighs.

Advantageously, and in order to obtain a remanent effect over time of the tensing agents on the homeostasis of the skin, it will be possible to apply the composition according to the invention, or the care kit, twice-weekly and, better still, daily, in the morning and/or the evening.

The effect of repeated application to the skin of tensing agents according to the invention on the mechanical properties of the skin, and in particular on the firmness, elasticity and extensibility of the skin, were also able to be confirmed and/or evaluated in vivo by instrumental devices such as those described below.

### The Torquemeter:

This apparatus aims to measure the variations in extensibility, in firmness/elasticity and in tonicity of the skin. The apparatus imposes a torsion in the plane of the skin for a given time; the skin is then subjected to a stretching which corresponds to its extensibility (Ue); after the arrest of the torsion, the skin regains its initial “shape”, and thus its tonicity (Ur) is evaluated. The firmness/elasticity of the skin corresponds to the ratio Ur/Ue (tonicity over extensibility).

### The Cutometer:

The cutometer is a suction apparatus composed of a cylinder from 1 to 3 mm in diameter which is applied to the skin. Drawing up the skin by means of a pump connected to the cylinder induces a vertical displacement of the skin, which makes it possible to evaluate the mechanical properties of the skin.

### The Densiscore:

The densiscore is an apparatus allowing the density of the skin to be measured. It subjects the skin locally to a mechanical stress which gives rise to folds, the number and amplitude of which are directly associated with the cutaneous density. Evaluation by trained experts of the profile of the skin subjected to the densiscore allows an evaluation of the density of the skin.

Moreover, the effect of repeated application to the skin of tensing agents according to the invention on the reorganization of the extracellular matrix may also be confirmed and/or evaluated in vivo by a technique of ultrasound echography.

The degradation and/or disorganization of the extra-cellular matrix at the level of the papillary dermis is a major cause of aging of the skin. It is partly responsible for the appearance of wrinkles and for the loss of density, firmness and extensibility of the skin. This degradation and/or disorganization of the extracellular matrix is even more visible in people who have mature (>40 years) or even very mature (>60-65 years) skin.

Ultrasound echography makes it possible to obtain 2D or 3D images of cutaneous tissues. The intensity of the echoes reflected provides information on the nature, density and organization of the constituents of the dermis. In particular it evidences differences between the superficial dermis or papillary dermis and the deep dermis or reticular dermis. This technique therefore makes it possible to evaluate the effect of the repeated application of tensing agents according to the invention on the reorganization and restructuring of the papillary dermis.

In the context of this method the composition may be, for example, a care composition or a makeup composition.

The invention likewise provides for the cosmetic use, in a composition comprising a physiologically acceptable medium, of at least one agent increasing the expression of mechanoreceptors in the cells of the skin, as an agent intended to sensitize the cells to the mechanical tensions induced by the topical application of a tensing agent.

In particular, the agent increasing the expression of mechanoreceptors in the cells of the skin is intended to potentialize and/or prolong the biomechanical effect of a tensing agent applied topically to the skin, at the level of the cells of the epidermis and/or the dermis.

The biomechanical effect of the tensing agent at the level of the cells of the epidermis and of the dermis is defined in particular by an improvement in the homeostasis of the skin, an increase in the thickness of the skin, an improvement in the radiance of the complexion, an improvement in the mechanical properties of the skin (e.g. firmness, elasticity, tonicity), and/or an improvement in the density of the skin, and/or an improvement in the regeneration and/or reorganization of the papillary dermis, and/or an improvement in the regeneration and/or reorganization of the extracellular matrix.

In particular, the agent increasing the expression of mechanoreceptors in the cells of the skin is intended to potentialize and/or prolong the effect of the tensing agent on the diminution of the processes of epidermal differentiation and/or the improvement of the regeneration and/or renewal of the skin.

The invention therefore likewise provides for the cosmetic use, in a composition comprising a physiologically acceptable medium, of at least one agent increasing the expression of mechanoreceptors in the cells of the skin in combination with a tensing agent, or for the use of a composition containing said combination, for promoting the improvement of the homeostasis of the skin, the increase of the thickness of the skin, the improvement of the radiance of the complexion, the density of the skin, the regeneration and/or the reorganization of the papillary dermis, the regeneration and/or the reorganization of the extra-cellular matrix and/or the improvement of the firmness, elasticity and/or tonicity of the skin.

According to a first embodiment the agent increasing the expression of mechanoreceptors in the cells of the skin and the tensing agent are present in a single composition.

According to an alternative embodiment, the agent increasing the expression of mechanoreceptors in the cells of the skin and the tensing agent are packaged in two separate compositions.

The agents increasing the expression of mechano-receptors in the cells of the skin and the tensing agents which can be used according to the present invention may be selected from the examples of agents described above in the description.

The invention will now be described with reference to the following examples, which are given by way of illustration and not of limitation. In these examples, unless indicated to the contrary, the amounts are expressed in weight percentages.

### FIGURES

FIG. 1: Example of mosaic deposition of Hybridur 875 (magnification X₃₀).

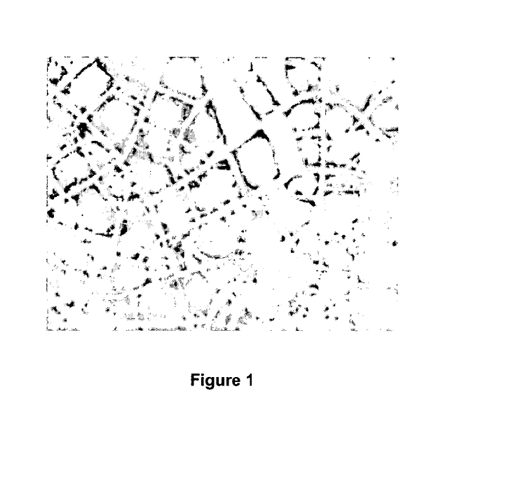

FIG. 2: Example of a curve of force as a function of displacement.

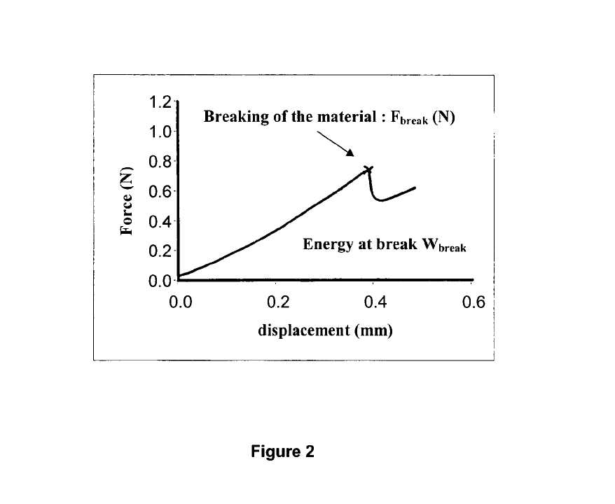

FIG. 3: Schematic representation of an electron micrograph showing the effect of Hybridur 875 on the reorganization of collagen fibrils in the extracellular matrix.

### EXAMPLES

### Example 1

### Demonstration of the Biological Effects of the Tensors

### a) Effect on the Differential Expression of Genes

The biological effects of the tensing agents were demonstrated after application to Episkin® reconstructed epidermides.

### Culture Conditions of Reconstructed Epidermides

The Episkin® reconstructed epidermides used were obtained at d 15. They were placed in a maintenance medium for 8 hours. They were then transferred to a DMEM/Ham F12 medium devoid of EGF, of pituitary extract and of foetal calf serum. The epidermides were placed in this medium for 24 hours in order to equilibrate.

### Preparation of the Tensor: Ethylenic Copolymer of Methyl Methacrylate/Methacrylic Acid Copolymer Type

### Step 1: Synthesis of the Polymer

A jacketed 2 l reactor was charged with 1 g of Trigonox 21S (tert-butyl peroxy-2-ethylhexanoate) and 200 g of methyl ethyl ketone. The mixture was heated at reflux for 1 h. After 1 h a mixture of 170 g of methyl methacrylate and 30 g of methacrylic acid was added dropwise over a time of 1 h. The colourless mixture became viscous. Heating was interrupted 6 h after the addition of the monomers.

Composition by NMR: methyl methacrylate 85.1%, methacrylic acid 14.9%

Mass by GPC in THF (polystyrene standards): Mw=98772; Mn=61261; Mw=105698 lp=1.7

### Step 2: Dispersion of the Polymer in Water

The above reaction mixture was admixed with 200 g of methyl ethyl ketone and heated to 60° C. 30.86 g of 2-amino-2-methylpropanol and 1200 g of water were added dropwise. The volatile solvents were evaporated by heating to 100° C. This gave a transparent yellow aqueous dispersion.

One hundred microlitres of an aqueous dispersion of this ethylenic copolymer were then applied to the Episkins in this culture medium and left in contact with the epidermides for 24 hours in a chamber thermostated at 37° C. and 40% relative humidity. At the end of this period, the epidermides were withdrawn and extracted for the cDNA array studies.

### Dedicated Analysis by Minichips

Gene expression analysis was performed by using standard DNA arrays containing 159 genes that were dedicated to the search and adapted for screening. These minichips were produced on a nylon support by fixing marker-specific cDNAs involved in the regulation of the physiology of keratinocytes and of the skin in general. The analysis is performed by an in-house optimized and miniaturized technique based on the use of mRNA and of labelling with phosphorus 33 (P33).

Schematically, the mRNAs of the cells were extracted and purified with the aid of trireagent, the mRNA of each culture is “reverse” transcribed using oligo dT and a P33-labelled deoxynucleotide triphosphate. Multiple cDNA “target” labelled sequences were therefore produced for each Episkin reconstructed epidermis. These targets were then hybridized, under optimized conditions, with cDNA “probes” in excess, fixed on the membranes. After washing, the quantity of labelled target is visualized by autoradiography and by direct counting on a PhosphorImager. The membranes are analysed by the Imagequant software.

The results are expressed in relative expression units. The levels of expression were corrected 1) for the average background noise present on each membrane and 2) for the differences in labelling intensity of the different probes used. This correction is carried out on the basis of differences in labelling intensity of the reference genes. The average of the counting results of “housekeeping gene” markers, whose expression is generally considered to be stable, was taken as a reference for quantifying, relatively, the expression of the other markers.

The significance limit was set at 180% of the untreated control for a stimulating effect and at 50% of the control for a repressive effect.

### Results

### Modulation of the Expression of Genes Involved in the Differentiation of Keratinocytes:

25 genes out of the 159 present on the dedicated mini-chips were modulated by the tensing agent. These genes intervene in the regulation of the physiology of keratinocytes and/or of fibroblasts.

The table below presents all of the results obtained on the effect of the tensors on the expression of these genes.

| | | | | | % variation |
|---|---|---|---|---|---|
| | | | | | of the |
| | | | | | expression |
| | | | | | of the genes |
| | | | | Growth | relative to |
| | | | | factors, | the |
| | | Differentiation | Metallo- | cytokines, | untreated |
| Abbreviation | Name of gene | markers | proteinases | receptors | control |
| AZGP1 | alpha-2- | | | | 21 |
| | glycoprotein 1, | | | | |
| | zinc | | | | |
| B2M | beta-2-micro- | | | | 486 |
| | globulin | | | | |
| CDSN | corneodesmosin | x | | | 51 |
| CST6 | cystatin 6 | x | | | 35 |
| CK1 | cytokeratin 1 | x | | | 980 |
| KRT19 | type I | x | | | 293 |
| | cytoskeletal 19 | | | | |
| | keratin | | | | |
| KRT2E | type II | x | | | 1163 |
| | cytoskeletal 2 | | | | |
| | epidermal keratin | | | | |
| | (KRT2E; KRT2A) | | | | |
| KRT6A | type II | x | | | 386 |
| | cytoskeletal 6 | | | | |
| | keratin: K6A | | | | |
| | keratin (KRT6A) | | | | |
| LOR | loricrin | x | | | 50 |
| NICE-1 | NICE-1 protein | x | | | 58 |
| CRBP1 | retinol-binding | x | | | 326 |
| | protein I | | | | |
| SPRL1B, | SPRL “small | x | | | 20 |
| XP5 | proline rich- | | | | |
| | like” protein | | | | |
| | (epidermal | | | | |
| | differentiation | | | | |
| | complex) 1B or | | | | |
| | skin-specific | | | | |
| | protein (XP5) | | | | |
| SPRL6A, | small proline- | x | | | 37 |
| LEP16 | rich-like | | | | |
| | (epidermal | | | | |
| | differentiation | | | | |
| | complex) 6A or | | | | |
| | SPRL6A; or late | | | | |
| | envelope protein | | | | |
| | 16 | | | | |
| KLK7 | kallikrein 7 | x | | | 56 |
| SBS | suprabasin (SBS) | x | | | 33 |
| ZYX | zyxin | x | | | 53 |
| MMP3 | matrix | | x | | 37 |
| | metalloproteinase | | | | |
| | 3 (MMP3) | | | | |
| IL6 | interleukin-6 | | | x | 340 |
| IL8 | interleukin-8 | | | x | 259 |
| TLR1 | oll-like receptor 1 | | | x | 434 |
| TGFB1 | transforming | | | x | 437 |
| | growth factor | | | | |
| | beta 1 | | | | |
| HMOX1 | heme oxygenase 1 | | | | 60 |
| HSPCA | heat shock | | | | 1083 |
| | 90 kDa protein 1 | | | | |
| MT1H | metallothionein | | | | 52 |
| | IH | | | | |
| MIF | macrophage | | | | 54 |
| | migration | | | | |
| | inhibitory factor | | | | |

In particular, the ethylenic copolymer tested reduced the expression of a number of proteins which make up the stratum corneum, such as corneodesmosin and loricrin, by a factor of two, and suprabasin by a factor of 3, which suggests that the copolymer diminishes the process of terminal differentiation.

The acrylic copolymer increases, moreover, the expression of a number of proteins of the intermediate filaments of the cytoskeleton, the cytokeratins, which are found in particular in foetal epitheliums and regenerative epitheliums. After 24 h of treatment, the expression of cytokeratins 1 is increased by a factor of 10 and the expression of cytokeratin 19 is increased by a factor of 3. These two cytokeratins, although present in adult epidermides, have been described as being expressed in many types of epithelial tissues, in particular in non-stratified epitheliums and also foetal epitheliums (Haake et al., Exp Cell Res., 1997 Feb. 25; 231(1): 83-95) Also increased is the expression of cytokeratin 2E/A, by a factor of 10: this cytokeratin 2 has been described as being expressed both in an adult epidermis and in a foetal epidermis. Finally, the expression of cytokeratin 6 is increased by a factor of 4. This cytokeratin 6 has been described as being overexpressed in regenerative epidermides, particularly in the course of cicatrization (Mazzalupo et al., 2003 February; 226(2): 356-65), which suggests that, in the course of the tensions provided by the application of the acrylic copolymer, the epidermides adopt features of regenerative epidermides.

The ethylenic copolymer according to the invention reduces the expression of complexes necessary for the process of differentiation of keratinocytes, such as SPRL, also called LEP10, by a factor of 5, and in the case of SPRL6 by a factor of 2.

In parallel with this, these results show that the copolymer increases the expression of CRBP1, which is involved in the cellular response to retinol, by a factor of 3, which suggests that the tensions are able to sensitize the cells to retinol.

### Modulation of the Expression of TGFb.

The expression of TGFb is increased by a factor of 4. This cytokine increases the expression and the organization of all of the fibrillar collagens and also of the plasminogen activator of type I, PAI1, and reduces the expression of a number of enzymes involved in the degradation of the extracellular matrix, the metalloproteinases.

In the course of the tensions caused by the acrylic latex it will be possible for the induced TGFb to diffuse to the level of the dermis and thus to induce tissue repair. The increase in the expression of TGFb by the tensions may be considered as a testament to the sensitivity of the cells to the tensions brought about by the acrylic latex tensor.

### Reduction in the Expression of Metalloproteinases:

The ethylenic copolymer reduces the expression of metalloproteinase 3, which is involved on the one hand in the migration of cells and on the other hand in the degradation of the extracellular matrix. The ethylenic copolymer thus inhibits the degradation of the extra-cellular matrix and plays a part in cell migration.

### Reduction of Zyxin:

The ethylenic copolymer reduced the expression of zyxin, which is known to be located at the level of the adhesion complexes and to play a part in the cell morphology.

### Increase in the Response of Cells to Environmental Stresses

The ethylenic copolymer increased the expression of the chaperone protein HSP90A by a factor of 10. HSP90A proteins play a fundamental part during the process of maturing of the proteins. They regulate the conformation of kinase or of transcription factors and, consequently, control their activity and their degradation.

The totality of these data show that the mechanical tensions applied via an effective amount of the tensing agent according to the invention are sensed by the keratinocytes as a stimulus which leads to a slowdown in the process of differentiation of the epidermis; the modulation of the expression of the genes cited above appears to show, moreover, that the epidermis acquires a regenerative epidermal phenotype. These results indicate that topical application of an effective amount of at least one tensing agent makes it possible to promote the homeostasis of the skin and thus to increase the thickness of the skin and/or improve the mechanical properties of the skin and/or promote the radiance of the complexion.

Moreover, the increase of the expression of HSP90 suggests that the tensions will reinforce the capacity of the epidermis to combat the alteration of the homeo-stasis of the skin that is induced by environmental stresses.

### Example 2

### Demonstration of an Effect of the Tensors on the Radiance of the Complexion

A serum is prepared comprising the ethylenic copolymer described in Example 1:

| | | |
|---|---|---|
| A- Water | 50.45 | g |
| Ammonium polyacryldimethyltauramide | 2.00 | g |
| (Hostacerine AMPS) | | |
| Preservatives | 0.85 | g |
| B- Ethylenic copolymer of Example 1 | 46.70 | g |
| (7% dispersion in water) | | |

The lightening and homogenizing effects and also the cosmetic aspects of this serum were evaluated on a panel of 6 subjects having normal/mixed skin. Following application of this serum in the evening for one month, a less poorly defined complexion was observed.

These results show that the biomechanical effect provided by the topical application of tensors to the skin, which is manifested in a reduction in epidermal differentiation and/or an increase in the regenerative power of the epidermis, makes it possible to enhance the appearance of the skin and in particular the radiance of the complexion.

### Example 3

### Mechanical Strength Testing of the Tensing Agents

The mechanical strength test consists in applying compressive stress to breaking point to the tensing agent under test at the surface of a flexible and deformable foam. The use of this foam support allows a substantial deformation to be imposed on the tensing agent under test, deposited at the surface, and therefore its breaking strength to be quantified.

The substrate is composed of a neoprene foam 13 mm in thickness.

The tensing agent, soluble or dispersible in water at a temperature ranging from 25° C. to 50° C. at a concentration of 7% by weight in water or at the maximum concentration by weight at which it forms in water at a temperature ranging from 25° C. to 50° C. a homogeneous medium visible with the naked eye, is deposited on this substrate to give, after drying for 24 h, a deposit with a thickness of 15 to 30 μm. The deposits were produced using a film-drawing device applying a wet film of 650 μm.

The mechanical compressive stress is exerted by means of a cylindrical punch with a diameter of 1 mm, the rate of displacement of the punch being 0.1 mm/s.

The test is carried out using a TA-XT2i texture analyser sold by Stable Micro System.

This gives a curve of force F (in N) as a function of displacement d (in mm), from which it is possible to determine the breaking point of the material (tensing agent) and the breaking energy W_{break} (J/m²) corresponding to the area under the curve F=f(d) at the breaking point F_{break} (N)

The totality of the results obtained are presented below:

| Compositions | d_{b} (mm) | W_{break} (J/m²) |
|---|---|---|
| Example A: Hybridur 875 (mosaic- | 0 | 0 |
| effect tensing agent of the | | |
| invention) | | |
| Example B: Eleseryl VGH8 | 0.4 | 40 |
| Example C: Flexan | 1.2 | 1600 |
| Example D: Avalure UR 405 | 1.7 | 1400 |
| Example E: Kytamer PCA | 1.5 | 3200 |

In particular, the mosaic-effect tensing agents according to the invention form deposits characterized advantageously by a breaking energy of between 0 and 20 J/m² (preferably equal to 0) and a breaking deformation of between 0 and 0.2 mm in this mechanical strength test.

### Example 4

### Demonstration of the Effect of the Tensors on the Reorganization of the Extracellular Matrix

### Principle of the Test

In order to define whether the application of tensing agents on the stratum corneum is able to induce modifications to the organization of the extracellular matrix, 100 μl of tensing agents, respectively Hybridur 875, sold by Air Products (at 15% by weight in water) and an acrylic tensor (ethylenic copolymer as prepared in Example 1, at 7% by weight in water), were applied to an Episkin® reconstructed skin model.

The reconstructed skin model, composed of human keratinocytes deposited on a support, often a dermis equivalent, and cultured under conditions such that they enter into a programme of differentiation leading ultimately to the formation of an epidermis equivalent, can be prepared according to the protocol described in Asselineau et al. (1987, Models in dermato., vol. III, Ed. Lowe & Maibach, 1-7).

Hybridur 875, sold by Air Products, is prepared in accordance with the description in patents U.S. Pat. No. 5,977,215 and U.S. Pat. No. 5,521,246.

The effect of these tensing agents on the dermis is observed after 2 h, 24 h and 48 h of application.

The analyses are carried out by two complementary imaging techniques: optical microscopy (multiphoton microscopy and optical microscopy in transmission of semi-fine sections) and electron microscopy (scanning electron microscopy).

Multiphoton microscopy allows rapid definition, without prior preparation of samples, of the actives which will exhibit an activity on the extracellular matrix, and on what timescale.

Its three-dimensional resolution makes it possible to determine the depth at which mechanical stimulation will cause dermal modifications.

When this first observation has been made, the samples are then analysed by scanning electron microscopy, which allows better resolution and hence allows the collagen fibrils to be individualized.

The modifications observed to the extracellular matrix are connected to modifications at the level of the fibroblasts by virtue of studies in optical microscopy in transmission of semi-fine sections.

### Results:

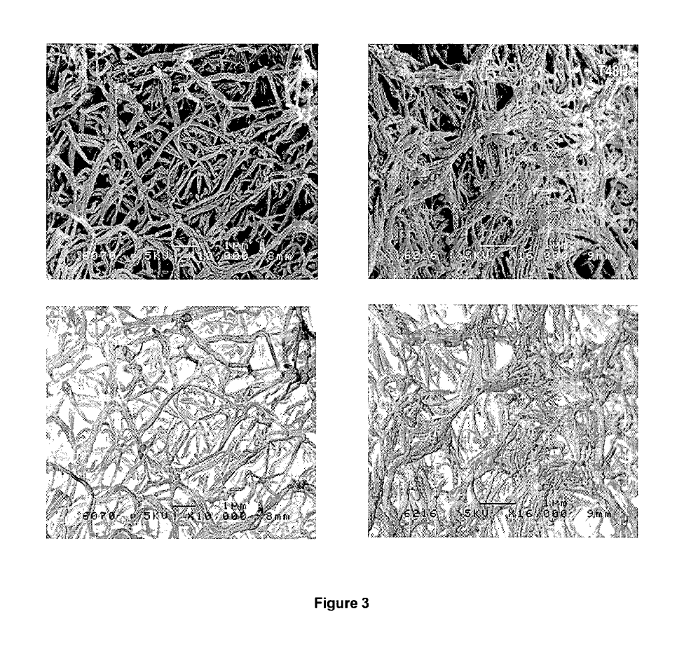

The observations by electron microscopy and photon microscopy show, as represented in FIG. 3, that the tensing agents tested induce reorganization of the collagen fibrils in the dermis after 48 h of application, and also a lengthening and an increase in the number of the fibroblasts.

The collagen fibrils of the extracellular matrix undergo association in fibrillar structure so as to form networks of greater density. This new organization may be linked to the synthesis of proteoglycans, such as decorin or else lumican, which are known to undergo association with a number of collagen molecules and thus to regroup them in a well-ordered network.

The mechanical stresses induced by the tensing agents have therefore stimulated the fibroblasts, leading to a reorganization of their cytoskeleton, and have done so from 48 h after application, which are therefore very short kinetics.

### Example 5

### Effect of the Combination between an Active increasing the Expression of Integrins and a Tensing Agent

The effect of the combination between an active increasing the expression of integrins (e.g.: manganese gluconate or hexapeptide Lys-Leu-Asp-Ala-Pro-Thr) and a tensing agent on the differential expression of genes involved in the homeostasis of the skin (e.g.: TGFb, keratin 19 and HSP90A) was evaluated by RT-Q-PCR on Episkin reconstructed epidermides, in comparison to the effect of the peptide and of the tensing agent per se.

The Episkin® reconstructed epidermides used are obtained at d 15. They are placed in a maintenance medium for 8 hours. They are subsequently transferred to a DMEM/Ham F12 medium devoid of EGF, of pituitary extract and of foetal calf serum. The epidermides are left in this medium for 24 hours to equilibrate.

The Episkin®s are then pretreated for 4 h and 24 h with 2 μg/ml manganese gluconate or 15 μg/ml hexapeptide Vinci02 (Lys-Leu-Asp-Ala-Pro-Thr) (Vincience), which are known to increase the number of mechanoreceptors (beta1 integrins) on the surface of the keratinocytes. After this pre-treatment, one hundred microlitres of an aqueous dispersion of polymer Hybridur 875, sold by Air Products (at 15% by weight in water), are applied to the Episkin®s in this culture medium and left in contact with the epidermides for 24 hours in a chamber thermostated at 37° C. and 40% relative humidity.

At the end of this period the epidermides are withdrawn and extracted for the RT-Q-PCR studies.

Other Episkin®s are treated under the same conditions but, respectively, with Hybridur 875 alone or with manganese gluconate/hexapeptide alone.

The effect of the products to be tested on the expression of the markers selected was evaluated by RT-Q-PCR, which was carried out on the basis of total RNAs extracted from the epidermides, in accordance with the following protocol:

The first step consists in carrying out a reverse transcription reaction.

This step requires prior treatment of the total RNAs in order to remove traces of potentially contaminating DNA by treatment with the system DNA-free (Ambion). The reverse transcription of the mRNAs to cDNAs takes place in the presence of the oligo(dT) primer and of the Superscript II enzyme (Gibco).

The PCRs (polymerase chain reactions) were carried out by quantitative PCR using the “Light Cycler” system (Roche Molecular Systems Inc.) and in accordance with the recommendations of the supplier. This analytical system allows rapid and high-performance PCR reactions to be carried out providing prior development of the conditions for analysis of the different primers. It is formed of two main components:
a thermocycler: optimized by virtue of the use of glass capillaries and of extremely rapid thermal transfers.
a fluorimeter: this allows the fluorescence intensity incorporated in the DNA to be measured continuously (detection at 521 nm).

The reaction mixture (10 μl final) introduced into capillaries for each sample is as follows:
2.5 μl of cDNA diluted 1/10.
primers of the different markers used
reaction mixture (Roche) containing the taq DNA polymerase enzyme, the SYBR marker and Green I (fluorophore which is intercalated into the double-stranded DNA in the course of the elongation step) and MgCl₂.

The incorporation of fluorescence into the amplified DNA is measured continuously in the course of the PCR cycles. This system enables curves to be obtained of measurement of the fluorescence as a function of the PCR cycles, and thus makes it possible to evaluate a relative expression value for each marker. The number of cycles is determined on the basis of the “exit” points of the fluorescence curves. For a given marker under analysis, the later the exit of a sample (high cycle number), the lower the initial number of copies of the mRNA. The RE (relative expression) value is expressed in arbitrary units in accordance with the following formula: (½ number of cycles)×10⁶.

### Example 6

### Cosmetic Compositions and Kits

### A—Compositions Containing the Ethylenic Copolymer of Example 1

### Oil-in-Water Emulsion

### Phase A

| | |
|---|---|
| Glyceryl stearate (and) PEG-100 stearate | 2.00 g |
| (Arlacel 165FL): | |
| Dimyristyl tartrate (and) cetearyl alcohol | 1.50 g |
| (and) C12-15 pareth-7 (and) PPG-25 laureth-25 | |
| (Cosmacol PSE): | |
| Cyclohexasiloxane: | 10.00 g |
| Stearyl alcohol: | 1.00 g |

### Phase B

| | |
|---|---|
| Water: | 41.5 g |
| Preservatives: | 0.75 g |
| Pentasodium ethylene diamine tetramethylene | 0.05 g |
| phosphate: | |
| Ammonium polyacryldimethyltauramide | 0.40 g |
| (Hostacerine AMPS): | |
| Xanthan gum (Rhodicare S): | 0.20 g |
| Zinc gluconate (Labcatal) | 1.8 g |

### Phase C

| | |
|---|---|
| Ethylenic copolymer of Example 1 (7% dispersion | 40.90 g |
| in water): | |

### Procedure:

heat phase B to approximately 75° C. and incorporate ammonium polyacryldimethyltauramide therein; stir until a homogeneous gel is obtained.
heat phase A to approximately 75° C.
produce the emulsion by incorporating phase A into phase B.
at 40-45° C., incorporate phase C and maintain stirring until cooling is complete.

The composition is applied daily to the face in order to promote the radiance of the complexion.

### W/O/W Triple Emulsion

Primary emulsion (A):

| | | |
|---|---|---|
| Water: | 10.20 | g |
| Polyglyceryl-4 isostearate, hexyl laurate and | 3.50 | g |
| cetyl PEG/PPG 10/1 dimethicone: | | |
| Cyclopentasiloxane: | 16.50 | g |
| Dimethicone: | 4.00 | g |
| Ethylenic copolymer of Example 1 (dispersion | 65.00 | g |
| at 7% in water): | | |
| Magnesium sulphate | 0.80 | g |

Multiple emulsion:

| | | |
|---|---|---|
| Primary emulsion (A): | 22.50 | g |
| Cyclopentasiloxane: | 3.50 | g |
| Apricot kernel oil: | 4.00 | g |
| Water: | 65.05 | g |
| Preservatives | 1.00 | g |
| Pentasodium ethylene diamine tetramethylene | 0.05 | g |
| phosphonate: | | |
| Alkyl acrylate copolymer: | 0.60 | g |
| Sodium hydroxide: | 0.30 | g |
| Peptide Gly-Pro-Gln-Gly-Pro-Gln (Collaxyl | 3 | g |
| sold by Vincience) | | |

### Procedure

### Preparation of the Primary Emulsion:

At ambient temperature and with stirring, the polyglyceryl-4 isostearate, the hexyl laurate, the cetyl PEG/PPG 10/1 dimethicone, the cyclopentasiloxane and the dimethicone are homogenized. With vigorous stirring, the water and the ethylenic copolymer of Example 1 are incorporated slowly.

### Preparation of the Triple Emulsion:

At ambient temperature and with stirring, the alkyl acrylate copolymer, the preservatives and the sequestrant (pentasodium ethylene diamine tetra-methylene phosphonate) are dispersed. The mixture is left to swell for approximately 45 minutes with stirring and then neutralized with sodium hydroxide. The primary emulsion is diluted with the cyclopentasiloxane and the apricot kernel oil, and then this mixture is incorporated slowly with stirring into the aqueous phase.

The composition is applied daily to the stomach and the thighs in order to improve the firmness and/or elasticity of the skin.

Water-in-Oil Emulsion

| | | |
|---|---|---|
| A- Polymethylcetyl dimethyl methylsiloxane, | 1.5 | g |
| oxyethylenated | | |
| Isostearate, polyglycerolated | 0.5 | g |
| Isohexadecane | 4 | g |
| Squalane | 1.85 | g |
| Dimethicone | 2.05 | g |
| Apricot kernel oil | 1.1 | g |
| Cyclopentasiloxane | 9 | g |
| Propylparaben | 0.15 | g |
| B- Water | 29.2 | g |
| Propylene glycol | 3 | g |
| Magnesium sulphate | 1.75 | g |
| Methylparaben | 0.2 | g |
| Preservative | 0.3 | g |
| Peptide Lys-Leu-Asp-Ala-Pro-Thr (Vinci 02 | 1.5 | g |
| sold by Vincience) | | |
| C- Ethylenic copolymer prepared according to | 40.9 | g |
| Example 1 (7% dispersion in water) | | |
| D- Nylon 12 | 3 | g |

Procedure:
homogenize phase A and phase B separately with stirring at ambient temperature.
produce the emulsion by incorporating phase B into phase A.
incorporate phases C and D with stirring.

### Serum

| | | |
|---|---|---|
| A- Water | 46.45 | g |
| Ammonium polyacryldimethyltauramide | 2.00 | g |
| (Hostacerine AMPS) | | |
| Preservatives | 0.85 | g |
| Copper gluconate (Labcatal) | 4 | g |
| B- Ethylenic copolymer prepared according to | 46.70 | g |
| Example 1 (7% dispersion in water) | | |

In an alternative embodiment the ethylenic copolymer is formulated in a separate composition for the purpose of preparing a skincare kit.

### B—Compositions containing Hybridur 875

Hybridur 875 is sold by Air Products and prepared in accordance with the description of patents U.S. Pat. No. 5,977,215 and U.S. Pat. No. 5,521,246.

### Oil-in-Water Emulsion

| | | |
|---|---|---|
| A- Glyceryl stearate (and) PEG-100 stearate | 2.00 | g |
| (Arlacel 165FL): | | |
| Dimyristyl tartrate (and) cetearyl alcohol | 1.50 | g |
| (and) C12-15 pareth-7 (and) PPG-25 laureth-25 | | |
| (Cosmacol PSE): | | |
| Cyclohexasiloxane: | 10.00 | g |
| Stearyl alcohol: | 1.00 | g |
| B- Water: | 66.10 | g |
| Preservatives: | 0.75 | g |
| Pentasodium ethylene diamine tetramethylene | 0.05 | g |
| phosphate: | | |
| Ammonium polyacryldimethyltauramide | 0.40 | g |
| (Hostacerine AMPS): | | |
| Xanthan gum (Rhodicare S): | 0.20 | g |
| Zinc gluconate | 1.00 | g |
| C- Hybridur 875 | 17.00 | g |

### Procedure:

heat phase B to approximately 75° C. and incorporate the ammonium polyacryldimethyltauramide therein; stir until a homogeneous gel is obtained.
heat phase A to approximately 75° C.
produce the emulsion by incorporating phase A into phase B.
at 40-45° C., incorporate phase C and maintain stirring until cooling is complete.

### W/O/W Triple Emulsion

Primary emulsion (A):

| | | |
|---|---|---|
| Water: | 58.20 | g |
| Polyglyceryl-4 isostearate, hexyl laurate and | 3.50 | g |
| cetyl PEG/PPG 10/1 dimethicone (ABILWE09): | | |
| Cyclopentasiloxane: | 16.50 | g |
| Dimethicone: | 4.00 | g |
| Hybridur 875 | 17.00 | g |
| Magnesium sulphate | 0.80 | g |

Multiple emulsion:

| | | |
|---|---|---|
| Primary emulsion (A): | 22.50 | g |
| Cyclopentasiloxane: | 3.50 | g |
| Apricot kernel oil: | 4.00 | g |
| Water: | 65.05 | g |
| Preservatives | 1.00 | g |
| Pentasodium ethylene diamine tetramethylene | 0.05 | g |
| phosphonate: | | |
| Alkyl acrylate copolymer (Pemulen TR1): | 0.60 | g |
| Sodium hydroxide: | 0.30 | g |
| Peptide Gly-Pro-Gln-Gly-Pro-Gln | 0.50 | g |

### Procedure

### Preparation of the Primary Emulsion:

At ambient temperature and with stirring, the polyglyceryl-4 isostearate, the hexyl laurate, the cetyl PEG/PPG 10/1 dimethicone, the cyclopentasiloxane and the dimethicone are homogenized. With vigorous stirring, the water and the Hybridur 875 are incorporated slowly.

### Preparation of the Triple Emulsion:

At ambient temperature and with stirring, the alkyl acrylate copolymer, the preservatives and the sequestrant (pentasodium ethylene diamine tetra-methylene phosphonate) are dispersed. The mixture is left to swell for approximately 45 minutes with stirring and then neutralized with sodium hydroxide. The primary emulsion is diluted with the cyclopentasiloxane and the apricot kernel oil, and then this mixture is incorporated slowly with stirring into the aqueous phase.

### W/O Emulsion

| | | |
|---|---|---|
| A- Polymethylcetyl dimethyl methylsiloxane, | 1.5 | g |
| oxyethylenated (Abil EM90) | | |
| Isostearate, polyglycerolated (Isolan GI34) | 0.5 | g |
| Isohexadecane | 4 | g |
| Squalane | 1.85 | g |
| Dimethicone | 2.05 | g |
| Apricot kernel oil | 1.1 | g |
| Cyclopentasiloxane | 9 | g |
| Propylparaben | 0.15 | g |
| B- Water | 54.10 | g |
| Propylene glycol | 3 | g |
| Magnesium sulphate | 1.75 | g |
| Methylparaben | 0.2 | g |
| Preservative | 0.3 | g |
| Peptide Lys-Leu-Asp-Ala-Pro-Thr | 0.50 | g |
| C- Hybridur 875 | 17.00 | g |
| D- Nylon 12 | 3 | g |

### Procedure:

homogenize phase A and phase B separately with stirring at ambient temperature.
produce the emulsion by incorporating phase B into phase A.
incorporate phases C and D with stirring.

### Serum

| | | |
|---|---|---|
| A- Water | 79.65 | g |
| Ammonium polyacryldimethyltauramide | 2.00 | g |
| (Hostacerine AMPS) | | |
| Preservatives | 0.85 | g |
| Copper gluconate | 0.50 | g |
| B- Hybridur 875 | 17.00 | g |

In an alternative embodiment the Hybridur 875 is formulated in a separate composition for the purpose of preparing a skincare kit.

1-35. (canceled)

36: A composition comprising:
at least one tensing agent, and
at least one peptide which increases the expression of mechanoreceptors in the cells of the skin, wherein
the at least one tensing agent and at least one peptide are in a physiological acceptable medium, and
the composition is for topical application to the skin.

37. The composition according to claim 36, wherein
the at least one peptide is selected from the group consisting of a mimetic peptide of fibronectin, a mimetic peptide of collagen and a mimetic peptide of laminin.

38: The composition according to claim 37, wherein the peptide is one selected from the group consisting of a fibronectin mimetic peptide of sequence (AA)ₙ-Leu-Asg-Ala-Pro-(AA)ₙ in which AA is any amino acid or derivative thereof and n is between 0 and 2; a collagen mimetic peptide of sequence (Gly-Pro-Gln)ₙ—NH₂ in which n is between 1 and 3; a laminin mimetic peptide of sequence X₁-Y-Phe-Thr-X₂-Ala-Thr-Z-Ile-X₃-Leu-X₄-Phe-Leu-X₅ in which X₁, X₂, X₃, X₄ and X₅=Arg, Lys or H is, Y=Asp or Glu and Z=Asn or Gln and derivatives thereof.

39: A composition comprising:
at least one tensing agent, and
at least one agent which increases the expression of mechanoreceptors in the cells of the skin, wherein
the at least one tensing agent and at least one agent which increases the expression of mechanoreceptors in the cells of the skin are in a physiological acceptable medium,
the composition is for topical application to the skin,
the at least one agent which increases the expression of mechanoreceptors in the cells of the skin is selected from the group consisting of a zinc salt, a copper salt, a manganese salt, a derivative thereof and a mixture thereof.

40: The composition according to claim 39, wherein
the at least one agent which increases the expression of mechanoreceptors in the cells of the skin is selected from the group consisting of a zinc gluconate and a manganese gluconate, and
when the at least one agent which increases the expression of mechanoreceptors in the cells of the skin is a zinc gluconate, the tensing agent is not a colloidal particle,
when the at least one agent which increases the expression of mechanoreceptors in the cells of the skin is a manganese gluconate, the tensing agent is not a graft polymer or a mixed silicate.

41: The composition according to claim 36, wherein a relative percent by weight composition of the at least one agent which increases the expression of mechanoreceptors in the cells of the skin is from 0.01% to 20%.

42: The composition according to claim 36, wherein the tensing agent is one selected from the group consisting of a synthetic polymer, an animal protein, a vegetable protein, a polysaccharide of natural origin in microgel form, a polysaccharide of natural origin in non-microgel form, a mixed silicate, a colloidal particle of an inorganic filler and a mixture thereof.

43: The composition according to claim 42, wherein the tensing agent is a synthetic polymer comprising an interpenetrating polymer network.

44: The composition according to claim 43, wherein the interpenetrating polymer network comprises a polyurethane polymer and an acrylic polymer.

45: The composition according to claim 36, wherein a percent by weight relative to the total weight of the composition of the tensing agent ranges from 0.1% to 30%.

46: A skincare kit comprising:
a first composition comprising, in a physiologically acceptable medium, at least one agent which increases the expression of mechanoreceptors in cells of a skin; and
a second composition comprising, in a physiologically acceptable medium, at least one tensing agent.

47: A method for cosmetic treatment of skin comprising:
applying to the skin at least one composition comprising:
at least one peptide which increases the expression of mechanoreceptors in cells of the skin; and
at least one tensing agent.

48: A method for cosmetic treatment of skin comprising:
applying to the skin at least one composition comprising in a physiologically acceptable medium:
at least one agent which increases the expression of mechanoreceptors in cells of the skin; and
at least one tensing agent;
wherein the cosmetic treatment is for at least one selected from the group consisting of an improvement of the homeostasis of the skin, an increase in a thickness of the skin, an improvement in a radiance of a complexion, an increase in a density of the skin, a regeneration of a papillary dermis, a reorganization of the papillary dermis, a regeneration of a extra-cellular matrix, a reorganization of the extra-cellular matrix, an improvement of a firmness of the skin, an improvement of an elasticity of the skin and an improvement in a tonicity of the skin.

49: The method according to claim 48, wherein the at least one agent which increases the expression of mechanoreceptors in cells of the skin and the at least one tensing agent are present in a single composition.

50: The method according to claim 48, wherein the at least one agent which increases the expression of mechanoreceptors in cells of the skin and the at least one tensing agent are present in two separate compositions.
